SEQUENCE LISTING (80 sequences)

SEQ ID NO: 1 - protein, 119 aa, synthetic construct
EVQLLESGGGLVQPGGSLRLSCAASGFPFGNYLMNWVRQAPGKGLEWVSVISGGGVSTYYADSVKGRFTISRDNSKNTLYLQMNSLRAEDTAVYYCARGPSSWPAFDVWGQGTLVTVSS

SEQ ID NO: 2 - protein, 125 aa, synthetic construct
EVQLLESGGGLVQPGGSLRLSCAASGFTFSIHPMTWVRQAPGKGLEWVSLISTGGGSTYYADSVKGRFTISRDNSKNTLYLQMNSLRAEDTAVYYCASASVLGSGGYWSPGMDYWGQGTLVTVSS

SEQ ID NO: 3 - protein, 124 aa, synthetic construct
EVQLVQSGAEVKKPGATVKISCKASGYSLSDHYMHWVQQAPGKGLEWMGRIDPHSGATKYDPKFQGRVTITADTSTDTAYMELSSLRSEDTAVYYCARSGPVHYYGSSYVMDYWGQGTTVTVSS

SEQ ID NO: 4 - protein, 124 aa, synthetic construct
EVQLVQSGAEVKKPGATVKISCKASGYSLSDHYMHWVQQAPGKGLEWMGRIDPHSGATKYDPKFQGRVTITADTSTDTAYMELSSLRSEDTAVYYCARSGPVYHYGSSYVMDYWGQGTTVTVSS

SEQ ID NO: 5 - protein, 124 aa, synthetic construct
EVQLVQSGAEVKKPGATVKISCKASGYILTDHYMHWVQQAPGKGLEWMGRIDPHSGGIKYDPKFQGRVTITADTSTDTAYMELSSLRSEDTAVYYCARSGPVYHYGSSYVMDYWGQGTTVTVSS

SEQ ID NO: 6 - protein, 107 aa, synthetic construct
DIQMTQSPSSLSASVGDRVTITCRASETISNFLNWYQQKPGKAPKLLIYTTSSLQRGVPSRFSGSGSGTDFTLTISSLQPEDFATYYCQQSYSVPWTFGGGTKVEIK

SEQ ID NO: 7 - protein, 107 aa, synthetic construct
DIQMTQSPSSLSASVGDRVTITCRASQSIDRFLNWYQQKPGKAPKLLIYGASNLPSGVPSRFSGSGSGTDFTLTISSLQPEDFATYYCQQSFSIPWTFGGGTKVEIK

SEQ ID NO: 8 - protein, 112 aa, synthetic construct
DIVMTQSPDSLAVSLGERATINCKSGQSVFYSSNNKNYLAWYQQKPGQPPKLLIYWASTRESGVPDRFSGSGSGTDFTLTISSLQAEDVAVYYCQQYLYSWTFGQGTKLEIK

SEQ ID NO: 9 - protein, 112 aa, synthetic construct
DIVMTQSPDSLAVSLGERATINCKSGQSVFYSSNNKNYLAWYQQKPGQPPKLLIYWADTRESGVPDRFSGSGSGTDFTLTISSLQAEDVAVYYCQQYLYSWTFGQGTKLEIK

SEQ ID NO: 10 - protein, 8 aa, synthetic construct
GFPFGNYL

SEQ ID NO: 11 - protein, 8 aa, synthetic construct
ISGGGVST

SEQ ID NO: 12 - protein, 12 aa, synthetic construct
ARGPSSWPAFDV

SEQ ID NO: 13 - protein, 6 aa, synthetic construct
ETISNF

SEQ ID NO: 14 - skipped

SEQ ID NO: 15 - protein, 9 aa, synthetic construct
QQSYSVPWT

SEQ ID NO: 16 - protein, 8 aa, synthetic construct
GFTFSIHP

SEQ ID NO: 17 - protein, 8 aa, synthetic construct
ISTGGGST

SEQ ID NO: 18 - protein, 18 aa, synthetic construct
ASASVLGSGGYWSPGMDY

SEQ ID NO: 19 - protein, 6 aa, synthetic construct
QSIDRF

SEQ ID NO: 20 - skipped

SEQ ID NO: 21 - protein, 9 aa, synthetic construct
QQSFSIPWT

SEQ ID NO: 22 - protein, 8 aa, synthetic construct
GYSLSDHY

SEQ ID NO: 23 - protein, 8 aa, synthetic construct
IDPHSGAT

SEQ ID NO: 24 - protein, 17 aa, synthetic construct
ARSGPVHYYGSSYVMDY

SEQ ID NO: 25 - protein, 17 aa, synthetic construct
ARSGPVYHYGSSYVMDY

SEQ ID NO: 26 - protein, 12 aa, synthetic construct
QSVFYSSNNKNY

SEQ ID NO: 27 - skipped

SEQ ID NO: 28 - protein, 8 aa, synthetic construct
QQYLYSWT

SEQ ID NO: 29 - protein, 8 aa, synthetic construct
GYILTDHY

SEQ ID NO: 30 - protein, 8 aa, synthetic construct
IDPHSGGI

SEQ ID NO: 31 - skipped

SEQ ID NO: 32 - protein, 8 aa, synthetic construct
GYTFTSYW

SEQ ID NO: 33 - protein, 8 aa, synthetic construct
IDPSDSYT

SEQ ID NO: 34 - protein, 7 aa, synthetic construct
ARSPFDY

SEQ ID NO: 35 - protein, 8 aa, synthetic construct
IDPSNSYT

SEQ ID NO: 36 - protein, 8 aa, synthetic construct
GYTFNTYW

SEQ ID NO: 37 - protein, 10 aa, synthetic construct
GFSLSTFGMG

SEQ ID NO: 38 - protein, 7 aa, synthetic construct
IWWDADK

SEQ ID NO: 39 - protein, 11 aa, synthetic construct
ARIGRNYYFDY

SEQ ID NO: 40 - protein, 12 aa, synthetic construct
QTLLDSTNQKNY

SEQ ID NO: 41 - protein, 9 aa, synthetic construct
QQYYTYPLT

SEQ ID NO: 42 - protein, 6 aa, synthetic construct
QNVGTN

SEQ ID NO: 43 - skipped

SEQ ID NO: 44 - protein, 9 aa, synthetic construct
QQYNSYPFT

SEQ ID NO: 45 - protein, 9 aa, synthetic construct
QQHNSYPFT

SEQ ID NO: 46 - protein, 6 aa, synthetic construct
QDVSTA

SEQ ID NO: 47 - protein, 9 aa, synthetic construct
HQHNTYPFT

SEQ ID NO: 48 - protein, 119 aa, synthetic construct
QVTLKESGPGILQPSQTLSLTCSFSGFSLSTFGMGVGWIRQPSGKGLEWLAHIWWDADKYSTPALKSRLTISKDTSKNQVFLKIANVDTADTATYYCARIGRNYYFDYWGQGTTLTVSS

SEQ ID NO: 49 - protein, 114 aa, synthetic construct
QVQLQQPGTELVRPGTSVKMSCKASGYTFTSYWMHWVKQRPGQGLEWIGVIDPSDSYTQYNQKFKGKATLTVDTSSSTAYMQLSSLTSEDSAVYYCARSPFDYWGQGTTLTVSS

SEQ ID NO: 50 - protein, 114 aa, synthetic construct
QVQLQQPGAELVRPGTSVKLSCKASGYTFTSYWMHWVKERPGQGLEWIGVIDPSDSYTNYNQKFKDKATLTVDTSSSTAYMQLSSLTSEDSAVYYCARSPFDYWGQGTTLTVSS

SEQ ID NO: 51 - protein, 114 aa, synthetic construct
QVQLQQPGAEVVMPGASVKLSCKASGYTFTSYWMHWVKQRPGQGLEWIGVIDPSDSYTQYNQKFKGKATLTVDTSSSTAYMQLSSLTSEDSAVYYCARSPFDYWGQGTTLTVSS

SEQ ID NO: 52 - protein, 114 aa, synthetic construct
EVQLQQPGAELVRPGTSVKLSCKASGYTFTSYWMHWVKQRPGQGLEWIGVIDPSDSYTQYNQKFKGKATLTVDTSSSTAYMQLSSLTSEDSAVYYCARSPFDYWGQGTTVTVSS

SEQ ID NO: 53 - protein, 114 aa, synthetic construct
QVQLQQPGAELVKPGASVKMSCKASGYTFTSYWMHWVKQRPGQGLEWIGVIDPSNSYTIYNQKFKGKATLTVDTSSSTAYMQLSSLTSEDSAVYYCARSPFDYWGQGTTLTVSS

SEQ ID NO: 54 - protein, 114 aa, synthetic construct
EVQLQQPGAELVRPGTSVKLACKASGYTFNTYWMHWVNQRPGQGLEWIGVIDPSDSYTQYNQKFKGKATLTVDTSSSTAYMQLSSLTSEDSAVYYCARSPFDYWGQGTTLTVSS

SEQ ID NO: 55 - protein, 113 aa, synthetic construct
DIVMTQSPSSLAVSVGEKVTLTCKSSQTLLDSTNQKNYLAWYQQIPGQSPKLLIYWASTRESGVPDRFTGSGSGTDFTLTISSVKAEDLTVYYCQQYYTYPLTFGAGTKLELK

SEQ ID NO: 56 - protein, 107 aa, synthetic construct
DIVMTQSQKFMSTSVGDRVSVTCKASQNVGTNVAWYQQKPGQSPKALIYSASYRYSGIPDRFTGSGSGTDFTLTISNVQSEDLADFFCQQYNSYPFTFGGGTKLEIK

SEQ ID NO: 57 - protein, 107 aa, synthetic construct
DIQMTQSQKFMSTSVGDRVSVTCKASQNVGTNVAWYQQKPGQSPKALIYSASYRYSGVPDRFIGSGSGTDFTLTIRNVQSEDLAEYFCQQHNSYPFTFGGGTKLEIK

SEQ ID NO: 58 - protein, 107 aa, synthetic construct
DIQMNQSHKFMSTSVGDRVSITCKASQDVSTAVAWYQQKPGQSPKALIYSASNRYSGVPDRFIGSGSGTDFTLTIRNVQSEDLAEYFCQQHNSYPFTFGGGTKLEMK

SEQ ID NO: 59 - protein, 107 aa, synthetic construct
DIVMTQSHKFMSTSVGDRVSITCKASQDVSTAVAWYQQKPGQSPKALIYSASYRYSGVPDRFTGSGSGTDFTLTISNVQAEDLAVYYCHQHNTYPFTFGGGTKLEIK

SEQ ID NO: 60 - protein, 107 aa, synthetic construct
DVVMTQSQKFMSTSVGDRVSVTCKASQNVGTNVAWYQQKPGQSPKALIYSASYRYSGVPDRFTGSGSGTDFTLTIRNVQSEDLAEYFCQQHNSYPFTFGGGTKLEIK

SEQ ID NO: 61 - protein, 107 aa, synthetic construct
DVQMTQSQKFMSTSVGDRVSVTCKASQNVGTNVAWYQQKPGQSPKALIYSASYRYSGVPDRFIGSGSGTDFTLTISNVQSEDLAEYFCQQHNSYPFTFGGGTKLEIK

SEQ ID NO: 62 - protein, 18 aa, Homo sapiens
RFRVTQLPNGRDFHMSVV

SEQ ID NO: 63 - protein, 288 aa, Homo sapiens
MQIPQAPWPVVWAVLQLGWRPGWFLDSPDRPWNPPTFSPALLVVTEGDNATFTCSFSNTSESFVLNWYRMSPSNQTDKLAAFPEDRSQPGQDCRFRVTQLPNGRDFHMSVVRARRNDSGTYLCGAISLAPKAQIKESLRAELRVTERRAEVPTAHPSPSPRPAGQFQTLVVGVVGGLLGSLVLLVWVLAVICSRAARGTIGARRTGQPLKEDPSAVPVFSVDYGELDFQWREKTPEPPVPCVPEQTEYATIVFPSGMGTSSPARRGSADGPRSAQPLRPEDGHCSWPL

SEQ ID NO: 64 - protein, 15 aa, synthetic construct
GGGGSGGGGSGGGGS

SEQ ID NO: 65 - protein, 8 aa, synthetic construct
GFNIKDTY

SEQ ID NO: 66 - protein, 8 aa, synthetic construct
IDPANGDR

SEQ ID NO: 67 - protein, 17 aa, synthetic construct
ARSGPVYYYGSSYVMDY

SEQ ID NO: 68 - protein, 8 aa, synthetic construct
GYPFTAHY

SEQ ID NO: 69 - protein, 8 aa, synthetic construct
GYTFPASY

SEQ ID NO: 70 - protein, 8 aa, synthetic construct
GYIFTDHF

SEQ ID NO: 71 - protein, 8 aa, synthetic construct
GYAFSGSF

SEQ ID NO: 72 - protein, 8 aa, synthetic construct
GYTFTADF

SEQ ID NO: 73 - protein, 8 aa, synthetic construct
IDPSSGGP

SEQ ID NO: 74 - protein, 8 aa, synthetic construct
INPQSGGS

SEQ ID NO: 75 - protein, 8 aa, synthetic construct
VSPISGGA

SEQ ID NO: 76 - protein, 8 aa, synthetic construct
LNPESGGT

SEQ ID NO: 77 - protein, 17 aa, synthetic construct
ARASGYYGSGSYSQLDY

SEQ ID NO: 78 - protein, 17 aa, synthetic construct
AREGIGSGYYHGGAFDY

SEQ ID NO: 79 - protein, 17 aa, synthetic construct
ARTPYYYGSTAKLFFDY

SEQ ID NO: 80 - protein, 17 aa, synthetic construct
ARSQDVVRGVIAYHFDY

(19) United States
(12) Patent Application Publication
(10) Pub. No.: US 2024/0092909 A1
(43) Pub. Date: Mar. 21, 2024

(54) PD-1 AGONIST ANTIBODIES

(71) Applicant: iBio, Inc., Bryan, TX (US)

(72) Inventors: Matthew P. Greving, Rancho Santa Fe, CA (US); Gao Liu, Brisbane, CA (US); Cody Allen Moore, Del Mar, CA (US); Alexander Tomoaki Taguchi, San Diego, CA (US)

(21) Appl. No.: 18/466,911

(22) Filed: Sep. 14, 2023

Related U.S. Application Data
(60) Provisional application No. 63/375,676, filed on Sep. 14, 2022.
(60) Provisional application No. 63/515,448, filed on Jul. 25, 2023.

(51) Int. Cl. C07K 16/28

(52) U.S. Cl. CPC C07K 16/2818; C07K 2317/24; C07K 2317/565; C07K 2317/622

(57) ABSTRACT

Provided herein are PD-1 agonist antibodies which bind PD-1. The PD-1 agonist antibodies of the disclosure are useful for the treatment of autoimmune and inflammatory diseases through the promotion of PD-1 signaling. Also provided herein are methods of use for the PD-1 agonist antibodies.

### CROSS-REFERENCE TO RELATED APPLICATIONS

This application claims priority to U.S. Provisional Application Ser. No. 63/375,676, filed Sep. 14, 2022 and U.S. Provisional Application Ser. No. 63/515,448, filed Jul. 25, 2023 the entire contents of which are incorporated herein by reference.

### STATEMENT OF FEDERALLY FUNDED RESEARCH

Not applicable.

### INCORPORATION-BY-REFERENCE OF MATERIALS FILED ON COMPACT DISC

The contents of the electronic sequence listing (IBIO_1036P2_SL_ST26.xml; Size: 69,377 bytes; and Date of Creation: Sep. 12, 2022) is herein incorporated by reference in its entirety.

### BACKGROUND OF THE INVENTION

Programmed cell death protein 1 (PD-1) is a cell-surface receptor with a critical role as an immune checkpoint inhibitor. PD-1 belongs to the immunoglobulin superfamily and is known to be expressed on T cells, B cells, monocytes, natural killer T cells, and dendritic cells. The transmembrane protein, programmed death-ligand 1 (PD-L1), serves as a natural ligand to PD-1. The PD-1: PD-L1 interaction serves to suppress immune cells through the phosphorylation of cytoplasmic immunoreceptor tyrosine-based inhibitory motifs (ITIMs) which activate downstream signaling which can inhibit T cell activation, cytokine production, and can also promote apoptosis. PD-1 is therefore critical to the prevention of autoimmunity and an overstimulated immune response which can be harmful to the body if left unchecked.

The PD-1: PD-L1 checkpoint is well known to be exploited by cancerous cells which upregulate PD-L1 to escape detection, and thus many PD-1 antagonist agents and antibodies have been developed as oncology therapeutics to combat this exploitation. Despite the focus on PD-1 antagonist agents, there is a need for PD-1 agonist agents and antibodies in the treatment of a variety of autoimmune and inflammatory diseases, which ideally do not interfere with innate PD-1: PD-L1 interactions. Provided herein are such antibodies.

### SUMMARY OF THE INVENTION

Provided herein are PD-1 agonist antibodies that bind PD-1. As embodied and broadly described herein, an aspect of the present disclosure relates to a PD-1 agonist antibody, wherein the antibody comprises: a heavy chain variable domain (VH) complementarity determining region (CDR) 1 comprising the amino acid sequence of any one of the following SEQ ID NOs: 10, 16, 22, 29, 32, 36, 37; and a VH CDR2 comprising the amino acid sequence of any one of the following SEQ ID NOs: 11, 17, 23, 30, 33, 35, 38; and a VH CDR3 comprising the amino acid sequence of any one of the following SEQ ID NOs: 12, 18, 24, 25, 34, 39; and a light chain variable domain (VL) CDR1 comprising the amino acid sequence of any one of the following SEQ ID NOs: 13, 19, 26, 40, 42, 46; and a VL CDR2 comprising the amino acid sequence of any one of the following SEQ ID NOs: 14, 20, 27, 31, 43; and a VL CDR3 comprising the amino acid sequence of any one of the following SEQ ID NOs: 15, 21, 28, 41, 44, 45, 47. In one aspect, antibody comprises: a VH comprising the amino acid sequence of any one of the following SEQ ID NOs: 1-5, 48-54, and a VL comprising the amino acid sequence of any one of the following SEQ ID NOs: 6-9, 55-61. In another aspect, the antibody is a monoclonal antibody. In another aspect, the antibody is a full-length antibody. In another aspect, the antibody is an antibody fragment. In another aspect, the antibody is fused to an Fc domain of any one of the following: human IgG1, human IgG2, human IgG3, and human IgG4.

As embodied and broadly described herein, an aspect of the present disclosure relates to a method of treating a disease in a subject in need thereof, comprising administering to the subject a therapeutically effective amount of the antibody described hereinabove. In one aspect, the disease is an autoimmune disease. In another aspect, the disease is an inflammatory disease. In another aspect, the subject is human.

As embodied and broadly described herein, an aspect of the present disclosure relates to a tandem scFv-Fc PD-1 agonist antibody wherein said antibody comprises an scFv1 and an scFv2 binding site in tandem on each antibody arm and wherein said scFv1 and scFv2 are linked by a linker, optionally a flexible linker. In one aspect, the antibody has a total of four scFv binding sites in a single scFv-Fc formatted antibody. In another aspect, the scFv1 of each antibody arm comprises a first heavy chain variable domain (VH1) and a first light chain variable domain (VL1); and wherein the scFv2 of each antibody arm comprises a first heavy chain variable domain (VH2) and a first light chain variable domain (VL2). In another aspect, the VH1 region and the VH2 region each comprises the amino acid sequence of any one of SEQ ID NOS: 1-5 and 48-54, more preferably SEQ ID NO: 1 or 53; and wherein the VL1 region and the VL2 region each comprises the amino acid sequence of any one of SEQ ID NOS: 6-9 and 55-61, or preferably SEQ ID NO: 6 or 60. In another aspect, the linker comprises the following amino acid sequence: GGGGSGGGGSGGGGS (SEQ ID NO: 64).

As embodied and broadly described herein, an aspect of the present disclosure relates to a method of treating a disease in a subject in need thereof, comprising administering to the subject a therapeutically effective amount of the antibody of any one of claims 1-15. In one aspect, the disease is an autoimmune disease. In another aspect, the disease is an inflammatory disease. In another aspect, the subject is human.

As embodied and broadly described herein, an aspect of the present disclosure relates to a nucleic acid encoding the PD-1 agonist antibody described hereinabove. In one aspect, the nucleic acid sequences are selected from those having at least 95, 96, 97, 98, 99, or 100% sequence identity to SEQ ID NOS: 66 and 67, 68 and 69, 70 and 71, 72, and 73, 74, and 75, 76, and 77, 78 and 79, 80 and 81, 82 and 83, 84 and 85, 86 and 87, 88 and 89, or 90 and 91. In another aspect, the nucleic acid sequences are selected from those having at least 95, 96, 97, 98, 99, or 100% sequence identity to variable heavy chains selected from 92, 94, 96, 98, 100, 102, or 104; and a light chain selected from SEQ ID NOS:93, 95, 97, 99, 101, 103, 104, or 105.

As embodied and broadly described herein, an aspect of the present disclosure relates to a nucleic acid vector comprising the nucleic acid sequence described hereinabove. As embodied and broadly described herein, an aspect of the present disclosure relates to a host cell comprising the nucleic acid vector described hereinabove.

### BRIEF DESCRIPTION OF THE DRAWINGS

The patent or application file contains at least one drawing executed in color. Copies of this patent or patent application publication with color drawing(s) will be provided by the Office upon request and payment of the necessary fee. The present application can be understood by reference to the following description taking in conjunction with the accompanying figures.

FIG. 1A illustrates the PD-1 agonist epitope directed MEM-nanoparticle B-Cell activation discovery strategy, as further described in Example 1.

FIG. 1B demonstrates the MEM-nanoparticle valency and PD1AB6 binding via Coomassie blue and Surface Plasmon Resonance (SPR) binding respectively.

FIG. 1C illustrates the mouse immunization protocol with alternating doses of a MEM-nanoparticle and full-length PD-1, with a final combinatory boost.

FIG. 1D shows the binding of the resulting mouse serum to PD-1 measured by enzyme-linked immunosorbent assay (ELISA).

FIG. 2A shows PD-1 binding of antibodies generated from monoclonal hybridomas by ELISA.

FIG. 2B shows binding of the exemplary antibody 27A5 to PD-1 (left) and competitive PD-1 binding of 27A5 (or lack thereof) against PD1AB6 and Pembrolizumab (right).

FIG. 2C shows the resulting PD-1 binding avidity curves for 27A5 measured by SPR.

FIG. 2D demonstrates the PD-1 agonist (left) and antagonist (right) response by 27A5, measured by a checkpoint signaling assay.

FIG. 3A illustrates the full-length PD-1 and MEM-nanoparticle phage panning strategy of in vitro scFv library, as described in Example 3.

FIG. 3B demonstrates the resulting SPR binding screen of isolated antibodies generated by phage panning.

FIG. 3C demonstrates that the isolated antibodies generated by phage panning do not compete with Pembrolizumab for PD-1 binding.

FIG. 3D shows the SPR binding avidity and affinity curves for exemplary antibodies, generated by the phage panning strategy.

FIG. 4A shows PD-1 agonist curves of exemplary antibodies, measured by a checkpoint signaling assay.

FIG. 4B shows PD-1 antagonist curves of exemplary antibodies, measured by a checkpoint signaling assay.

FIG. 4C shows competitive PD-1 binding of exemplary antibodies against PD1AB6 (left) or Pembrolizumab (right) via SPR.

FIG. 4D shows the compiled avidity, affinity, and agonist EC50 values of exemplary antibodies generated by the phage panning strategy.

FIG. 5 shows PD-1 SPR binding curves of the resulting two exemplary antibodies.

FIG. 6A shows PD-1 agonist curves of exemplary antibodies measured by a checkpoint signaling assay.

FIG. 6B shows PD-1 antagonist curves of exemplary antibodies measured by a checkpoint signaling assay.

FIG. 7A illustrates the AI-model/Mammalian display antibody discovery strategy as described in Example 7.

FIG. 7B shows the resulting PD-1 SPR binding curves for exemplary antibodies.

FIG. 7C shows the thermal stability measurements for exemplary antibodies.

FIG. 8A demonstrates the complete blockade of PD1AB6 binding to PD-1 by exemplary antibodies.

FIG. 8B demonstrates the PD-1 agonist curves of the exemplary antibodies, measured by a checkpoint signaling assay.

FIG. 8C demonstrates the lack of PD-1 antagonism by the exemplary antibodies, measured by a checkpoint signaling assay.

FIG. 9 demonstrates PD-1 agonist activity by the exemplary antibodies, measured by human primary CD4 T-cell cytokine release and activation marker expression.

FIG. 10 demonstrates epitope binning for three exemplary clones that exhibit potent PD-1 agonist activity.

FIG. 11 illustrates an exemplary tandem scFv-Fc formatted antibody structure of the disclosure.

FIG. 12 demonstrates melting temps for tandem scFv-Fc formatted antibodies.

FIG. 13 demonstrates PD-1 agonist activity by exemplary tandem scFv-Fc format antibodies, measured by a checkpoint signaling assay.

FIG. 14 demonstrates lack of PD-1 antagonism by exemplary tandem scFv-Fc format antibodies, measured by a checkpoint signaling assay.

FIG. 15 demonstrates PD-1 agonist activity by exemplary tandem scFv-Fc format antibodies, measured by human primary CD4 T-cell cytokine release and activation marker expression.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

Provided herein are agonist antibodies that bind to PD-1 at a site not recognized by PD-L1. Also provided are methods of making and using such antibodies. These antibodies may be useful for downregulating an immune response in an individual. For example, in some embodiments, the antibodies may be used for treating diseases which involve autoimmunity and/or hyperinflammation.

Where elements are presented in a list format (e.g., in a Markush group), it should be understood that each possible subgroup of the elements is also disclosed, and that any one or more elements can be removed from the list or group.

It should be understood that, unless clearly indicated, in any method described or disclosed herein that includes more than one act, the order of the acts is not necessarily limited to the order in which the acts of the method are recited, but the disclosure encompasses exemplary embodiments in which the order of the acts is so limited.

The terms used throughout the specification are defined as follows unless otherwise limited in specific instances. As used in the specification and the claims, the singular forms “a,” “an,” and “the” include plural referents unless the context clearly dictates otherwise. All technical and scientific terms, acronyms, and abbreviates used in the specification and claims have the same meaning as commonly understood by one of ordinary skill in the art to which the disclosure pertains, unless defined or stated otherwise. All numerical ranges are inclusive of the values defining the range as well as all integer values in between, unless indicated or defined otherwise.

The term “antibody” as used herein throughout is used in the broadest sense and includes a monoclonal antibody, polyclonal antibody, human antibody, humanized antibody, non-human antibody, chimeric antibody, a monovalent antibody, an antibody fragment, and a tandem scFv-Fc antibody.

Antibody fragments of the disclosure retain PD-1 antigen binding specificity. Antibody fragments include antigen-binding fragments (Fab), variable fragments (Fv) containing VH and VL sequences, single chain variable fragments (scFv) containing VH and VL sequences linked together in one chain, single chain antibody fragments (scAb) or other antibody variable region fragments, such as retaining antigen binding specificity.

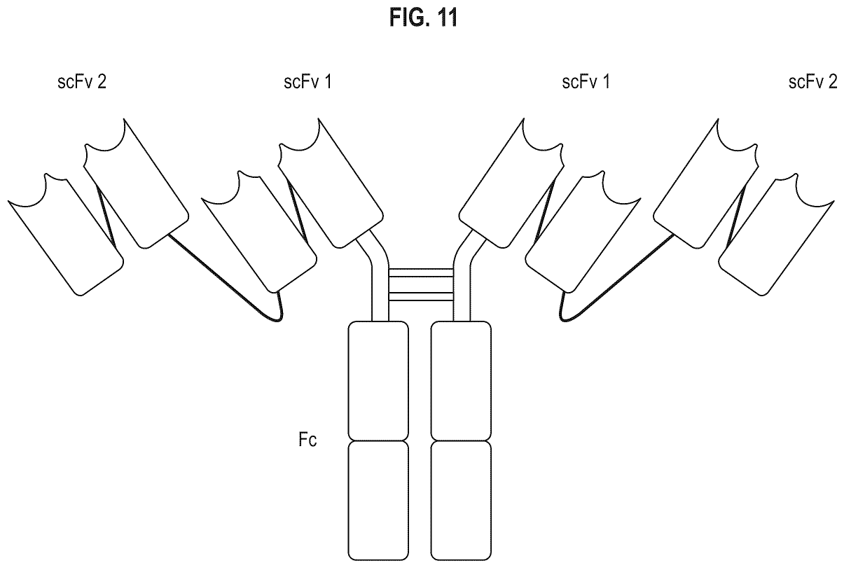

Tandem scFv-Fc antibodies of the disclosure are composed two or more scFv binding sites in tandem on each antibody arm, optionally linked by a linker, optionally a flexible linker, giving rise to a total of four or more scFv binding sites in a single scFv-Fc formatted antibody. FIG. 11 illustrates an exemplary tandem scFv-Fc formatted antibody structure of the disclosure.

The term “meso scale-molecule (MEM)” as used herein throughout includes engineered peptides and polypeptides between about 1 kDa and about 10 kDa. The term “MEM-nanoparticle” as used herein throughout includes MEMS which have been conjugated to a nanoparticle (e.g. ferritin nanoparticle).

As used herein, a “subject” may be a mammalian subject. Mammalian subjects include, humans, non-human primates, rodents, (e.g., rats, mice), lagomorphs (e.g., rabbits), ungulates (e.g., cows, sheep, pigs, horses, goats, and the like), etc. In some embodiments, the subject is a human. In some embodiments, the subject is a non-human primate, for example a cynomolgus monkey. In some embodiments, the subject is a companion animal (e.g. cats, dogs).

All publications, patents, and patent applications mentioned in this specification are herein incorporated by reference to the same extent as if each individual publication, patent, or patent application was specifically and individually indicated to be incorporated by reference.

I. PD-1 Agonist Antibodies

Provided herein are antibodies that bind to PD-1 and elicit an agonistic response. These antibodies are referred to herein as PD-1 agonist antibodies. A number of discovery strategies have been employed to obtain the exemplary antibodies of the disclosure, further discussed below.

The amino acid sequence of full-length human PD-1 is provided as SEQ ID NO: 63 (referencing UniProtKB ID Q15116).

(SEQ ID NO: 63)
MQIPQAPWPVVWAVLQLGWRPGWFLDSPDRPWNPPTESPALLVVTEGDN
ATFTCSFSNTSESFVLNWYRMSPSNQTDKLAAFPEDRSQPGQDCRFRVT
QLPNGRDFHMSVVRARRNDSGTYLCGAISLAPKAQIKESLRAELRVTER
RAEVPTAHPSPSPRPAGQFQTLVVGVVGGLLGSLVLLVWVLAVICSRAA
RGTIGARRTGQPLKEDPSAVPVFSVDYGELDFQWREKTPEPPVPCVPEQ
TEYATIVFPSGMGTSSPARRGSADGPRSAQPLRPEDGHCSWPL

In some embodiments, the PD-1 agonist antibodies specifically bind to the PD-1 epitope identified by the amino acid sequence provided as RFRVTQLPNGRDFHMSVV SEQ ID NO: 62.

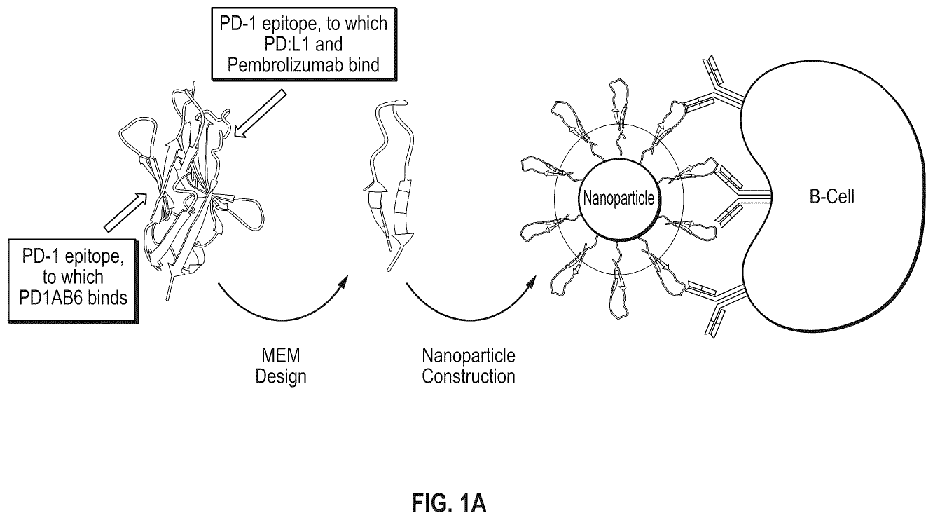

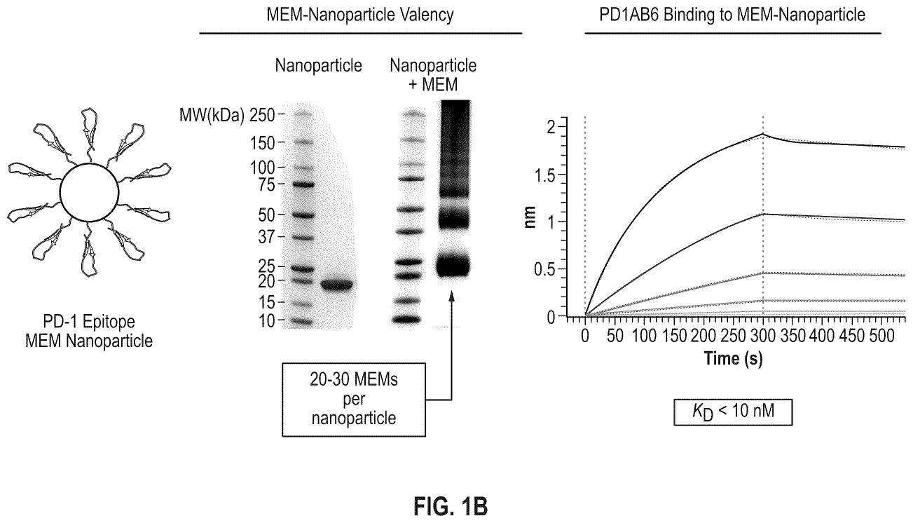

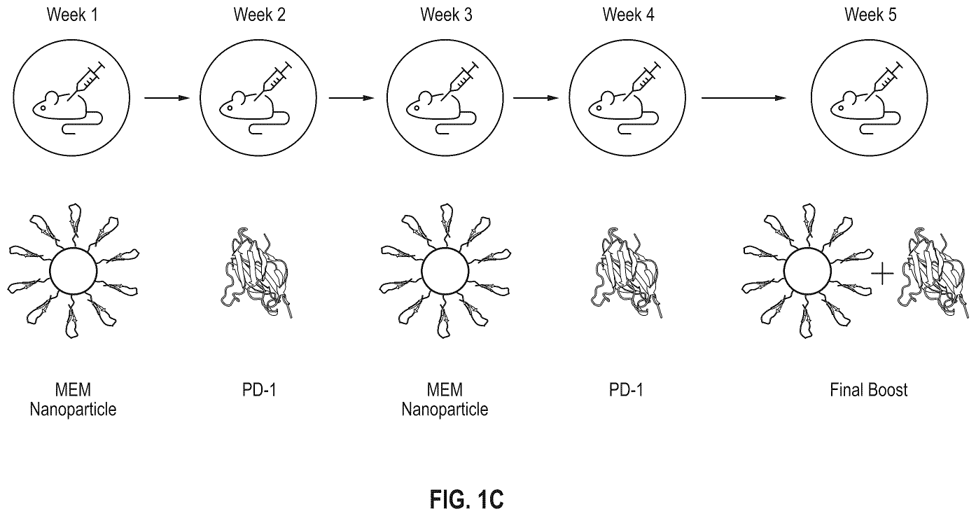

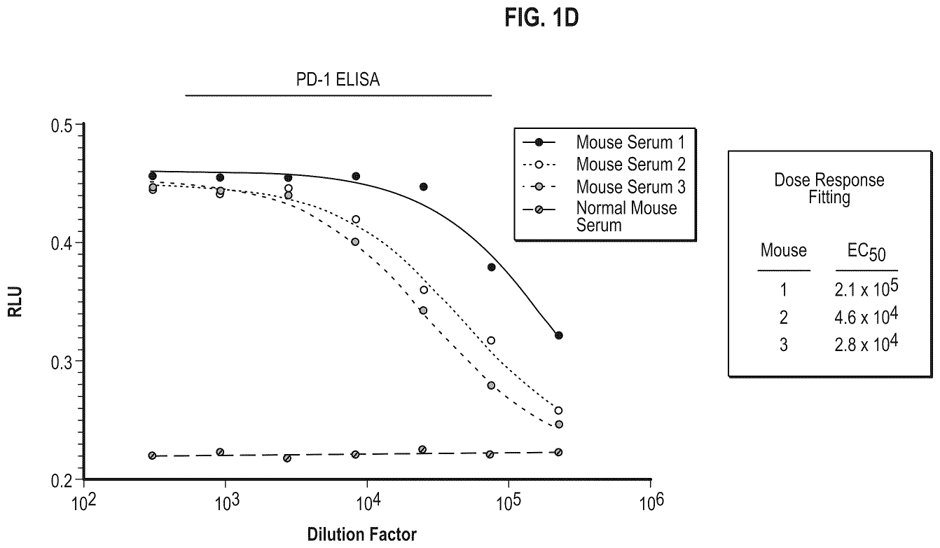

Referring to FIG. 1A, the MEMs of the disclosure are made to mimic the agonist epitope identified by SEQ ID NO: 62 and are subsequently used to screen for antibodies. The advantage of this approach is the ability to steer antibody discovery away from the PD-L1 binding site and toward the desired epitope.

In some embodiments, the MEM-nanoparticles along with full length PD-1 are used to immunize a subject in order to produce antibodies specific to the MEM epitope. Monoclonal hybridomas are then created to produce epitope specific PD-1 antibodies. In other embodiments, mouse serum is collected and used to produce in vitro scFv libraries. Followed by phage panning against full length PD-1 and the MEM-nanoparticle to isolate epitope specific clones. In other embodiments, antibodies are generated using phage panning of a naïve antibody library against full length PD-1 and the MEM-nanoparticle. In other embodiments, humanized PD-1 CDRs are generated from AI-model predictions based on reference antibodies. Mammalian display libraries are then created, and single cell sorted to select for epitope specific PD-1 antibodies.

The skilled artisan will recognize that antibodies which exhibit little or no binding to a target antigen can be described as having a low affinity, and a high equilibrium dissociation constant (KD) for the target antigen. The skilled artisan will also recognize that antibodies which exhibit little or no binding to a collective assembly of target antigenic epitopes can be described as having a low avidity, and a high equilibrium dissociation constant (KD) for the collective assembly of target antigenic epitopes.

In some embodiments, provided herein are PD-1 agonist antibodies having a binding affinity (KD) to PD-1 of about 5 μM to about 5 pM, about 1 μM to about 5 pM, about 0.5 μM to about 5 pM, about 0.1 μM to about 5 pM, about 50 nM to about 5 pM, about 10 nM to about 5 pM, about 5 nM to about 5 pM, about 1 nM to about 5 pM, about 0.5 nM to about 5 pM, about 0.1 nM to about 5 pM, about 50 pM to about 5 pM, about 10 pM to about 5 pM.

In some embodiments, PD-1 agonist antibodies have a binding avidity (KD) to PD-1 of about 500 nM to about 0.1 pM, about 100 nM to about 0.1 pM, about 50 nM to about 0.1 pM, about 10 nM to about 0.1 pM, about 5 nM to about 0.1 pM, about 1 nM to about 0.1 pM, about 0.5 nM to about 0.1 pM, about 0.1 nM to about 0.1 pM, about 50 pM to about 0.1 pM, about 10 pM to about 0.1 pM, about 5 pM to about 0.1 pM, about 1 pM to about 0.1 pM, about 0.5 pM to about 0.1 pM.

In some embodiments, PD-1 agonist antibodies have a half maximal effective concentration (EC50) to PD-1 of about 500 nM to about 0.001 nM, about 100 nM to about 0.001 nM, about 50 nM to about 0.001 nM, about 10 nM to about 0.001 nM, about 5 nM to about 0.001 nM, about 1 nM to about 0.001 nM, about 0.5 nM to about 0.001 nM, about 0.1 nM to about 0.001 nM, about 0.05 nM to about 0.001 nM, about 0.01 nM to about 0.001 nM, about 0.005 nM to about 0.001 nM.

The skilled artisan will recognize that binding specificity may be determined through a series of competition binding paradigms, in which a desired antibody demonstrates its ability to prevent binding of a known reference antibody to its target epitope at varying concentrations. In some embodiments, the reference PD-1: PD-L1 antagonist antibody is Pembrolizumab. In some embodiments, the reference agonist antibody, which binds the epitope recognized by SEQ ID NO: 62, is PD1AB6 (referencing patent number: U.S. Ser. No. 10/428,145B2). The skilled artisan will also recognize that PD1AB6 and Pembrolizumab may be utilized as control antibodies in agonist and antagonist assays.

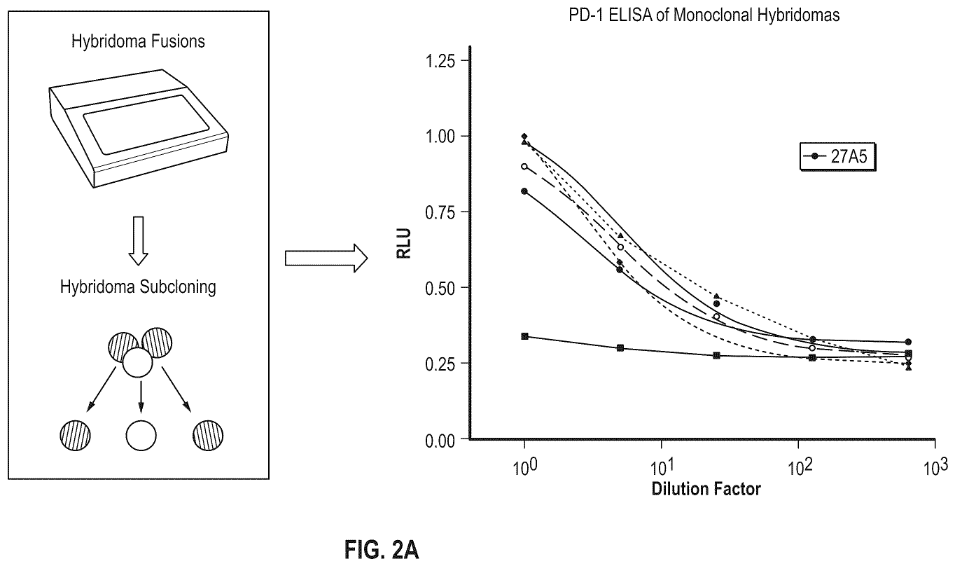

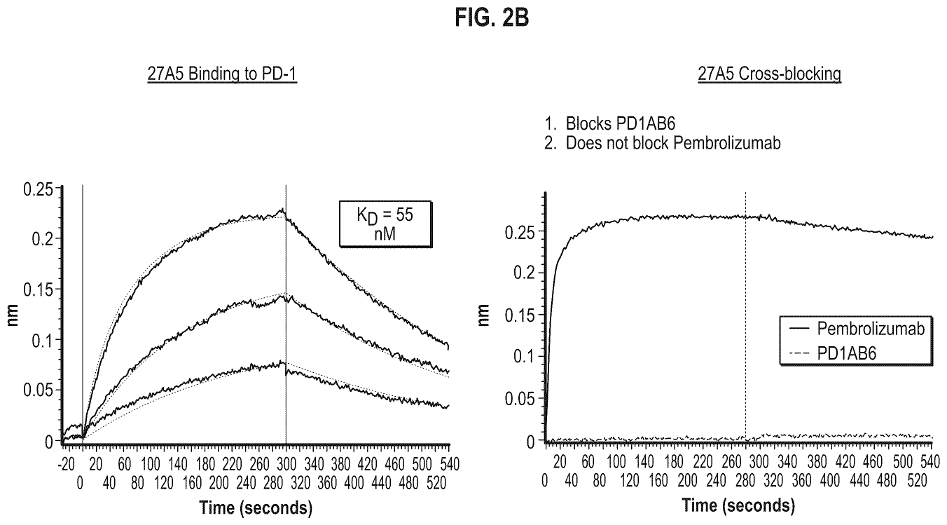

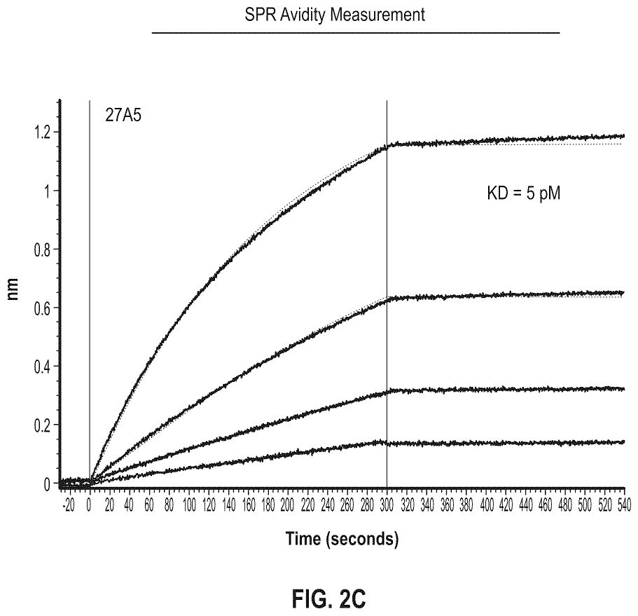

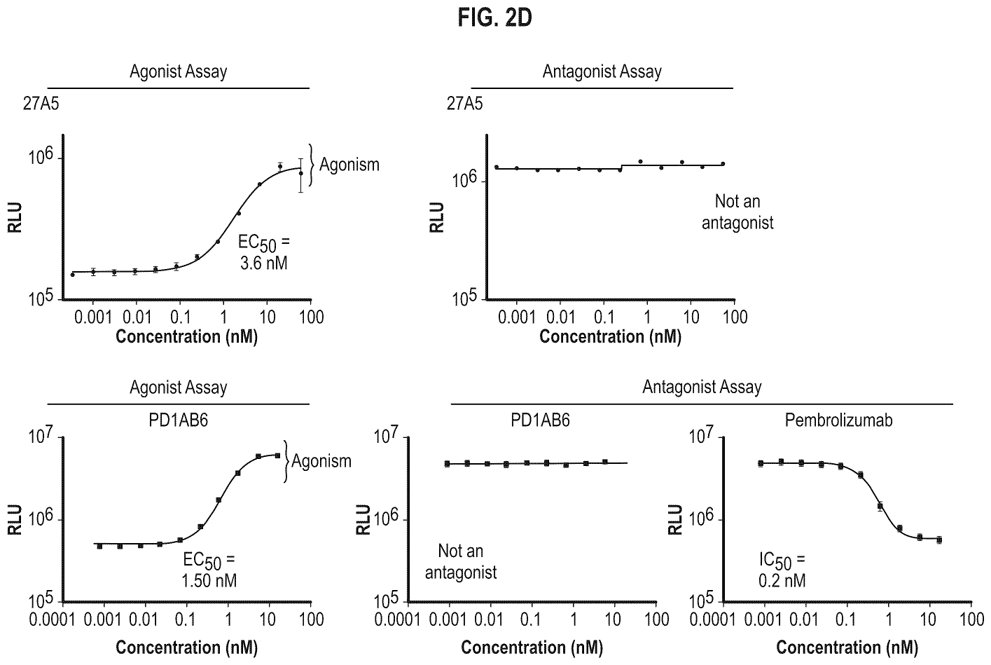

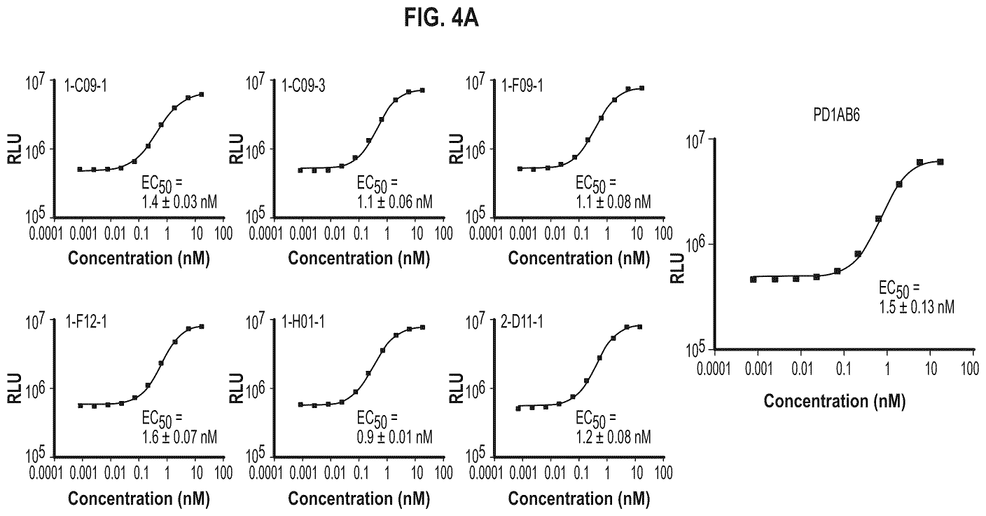

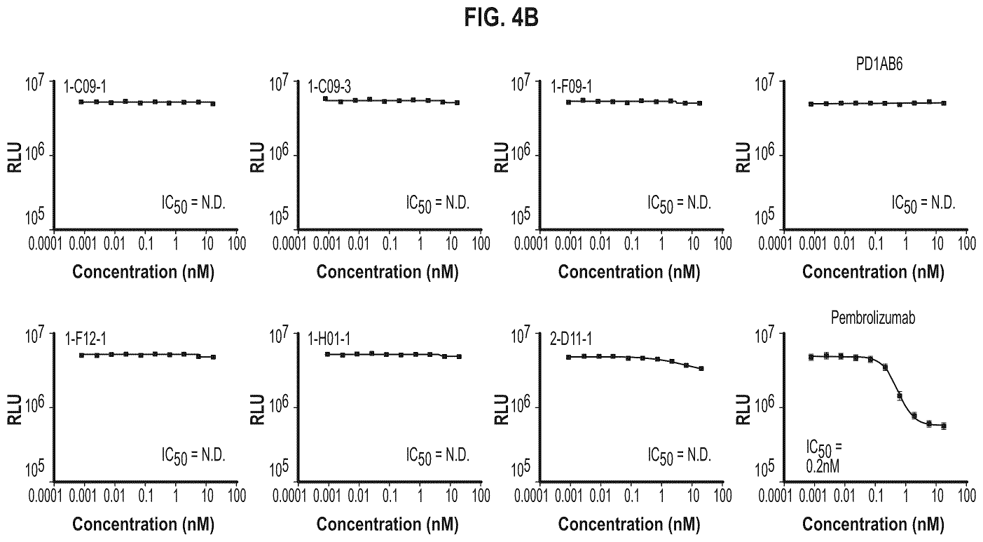

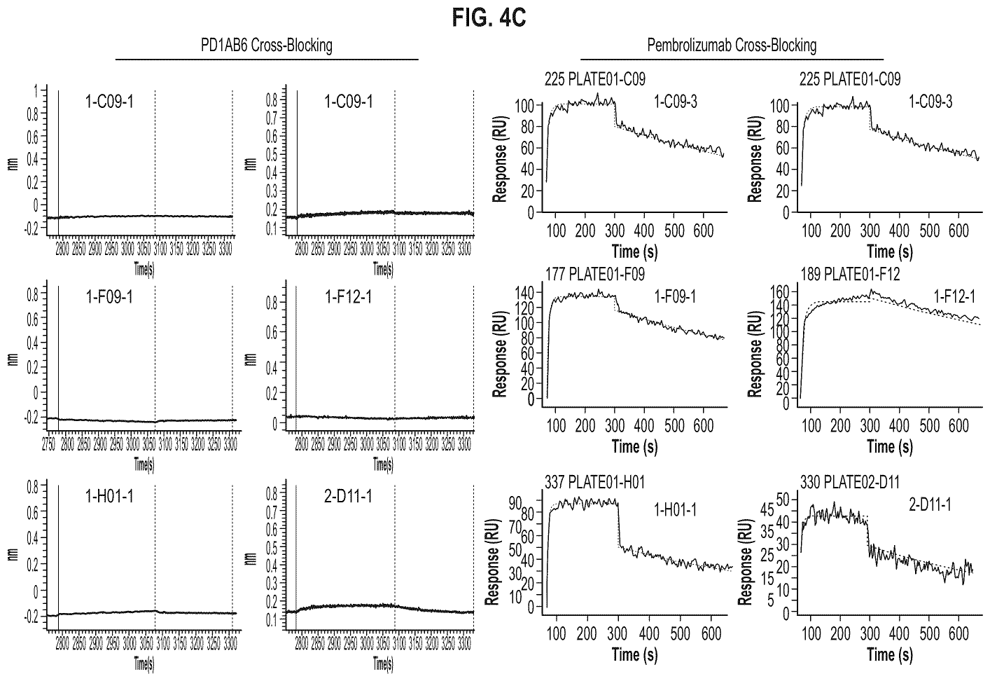

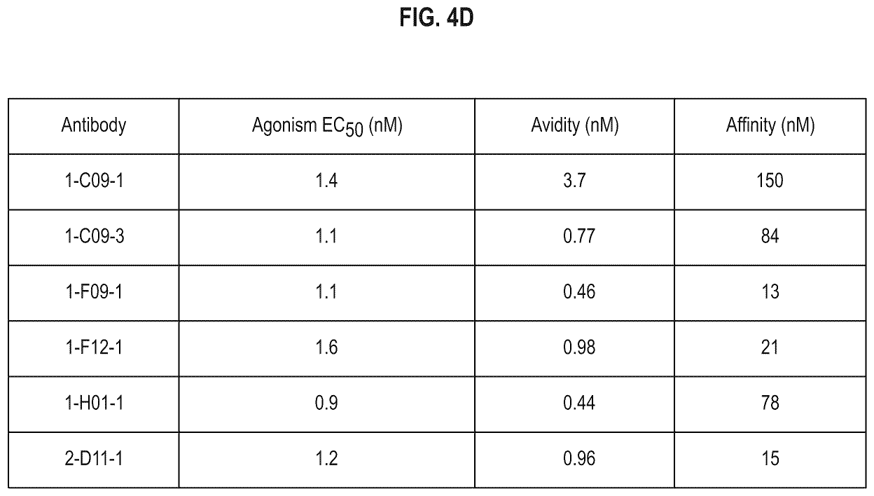

In some embodiments, the PD-1 agonist antibodies of the disclosure do not disrupt Pembrolizumab binding to PD-1. Exemplary antibodies of the disclosure that do not disrupt Pembrolizumab binding to PD-1 include antibodies 27A5, 1-C09-1, 1-C09-3, 1-F09-1, 1-F12-1, 1-H01-1, and 2-D11-1. FIGS. 2B and 4C. The PD-1 agonist antibodies of the disclosure disrupt PD1AB6 binding to the PD-1 epitope identified by the SEQ ID NO: 62.

In some embodiments, the PD-1 agonist antibody is a full length antibody (referring to an antibody with two heavy and two light chains attached to the Fc domain, giving a ‘Y’ shape). In some embodiments the Fc domain (or simply referred to as an Fc) is a human Fc domain. In some embodiments, the Fc domain of a PD-1 agonist antibody is from a human IgG1, human IgG2, human IgG3, or human IgG4.

A. Exemplary PD-1 Agonist Antibodies—CDR Sequences

Provided herein are sequences for exemplary PD-1 agonist antibodies of the disclosure. Included are complementarity determining region (CDR) sequences and the variable heavy and light domain sequences (VH, VL) that constitute the PD-1 antigen binding domains of the disclosure. The discovery of these antibodies is detailed in the Examples section.

As referred below, a light chain variable (VL) domain CDR1 region is referred to as CDR-L1; a VL CDR2 region is referred to as CDR-L2; a VL CDR3 region is referred to as CDR-L3; a heavy chain variable (VH) domain CDR1 region is referred to as CDR-H1; a VH CDR2 region is referred to as CDR-H2; and a VH CDR3 region is referred to as CDR-H3. Table 1 provides exemplary CDR combinations of antibodies of the disclosure.

TABLE 1
Exemplary PD-1 Agonist Antibody CDR Combinations

| Antibody | | | | | | |
|---|---|---|---|---|---|---|
| ID | CDR-H1 | CDR-H2 | CDR-H3 | CDR-L1 | CDR-L2 | CDR-L3 |
| 7 | GFPFGNY | ISGGGVS | ARGPSSW | ETISNF | TTS (SEQ | QQSYSVP |
| | L (SEQ ID | T (SEQ ID | PAFDV | (SEQ ID | ID NO: 14) | WT (SEQ |
| | NO: 10) | NO: 11) | (SEQ ID | NO: 13) | | ID NO: 15) |
| | | | NO: 12) | | | |
| 43 | GFTFSIHP | ISTGGGS | ASASVLG | QSIDRF | GAS (SEQ | QQSFSIP |
| | (SEQ ID | T (SEQ ID | SGGYWSP | (SEQ ID | ID NO: 20) | WT (SEQ |
| | NO: 16) | NO: 17) | GMDY | NO: 19) | | ID NO: 21) |
| | | | (SEQ ID | | | |
| | | | NO: 18) | | | |
| E2-B5- | GYSLSDH | IDPHSGA | ARSGPVH | QSVFYSSN | WAS (SEQ | QQYLYS |
| 6_201 | Y (SEQ ID | T (SEQ ID | YYGSSYV | NKNY | ID NO: 27) | WT (SEQ |
| | NO: 22) | NO: 23) | MDY (SEQ | (SEQ ID | | ID NO: 28) |
| | | | ID NO: 24) | NO: 26) | | |
| E2-B5- | GYSLSDH | IDPHSGA | ARSGPVY | QSVFYSSN | WAS (SEQ | QQYLYS |
| 7_201 | Y (SEQ ID | T (SEQ ID | HYGSSYV | NKNY | ID NO: 27) | WT (SEQ |
| | NO: 22) | NO: 23) | MDY (SEQ | (SEQ ID | | ID NO: 28) |
| | | | ID NO: 25) | NO: 26) | | |
| E2-B5- | GYSLSDH | IDPHSGA | ARSGPVY | QSVFYSSN | WAD (SEQ | QQYLYS |
| 7_211 | Y (SEQ ID | T (SEQ ID | HYGSSYV | NKNY | ID NO: 31) | WT (SEQ |
| | NO: 22) | NO: 23) | MDY (SEQ | (SEQ ID | | ID NO: 28) |
| | | | ID NO: 25) | NO: 26) | | |
| D4-A7- | GYILTDH | IDPHSGGI | ARSGPVY | QSVFYSSN | WAS (SEQ | QQYLYS |
| 7_201 | Y (SEQ ID | (SEQ ID | HYGSSYV | NKNY | ID NO: 27) | WT (SEQ |
| | NO: 29) | NO: 30) | MDY (SEQ | (SEQ ID | | ID NO: 28) |
| | | | ID NO: 25) | NO: 26) | | |
| 27A5 | GFSLSTFG | IWWDAD | ARIGRNY | QTLLDST | WAS (SEQ | QQYYTY |
| | MG (SEQ | K (SEQ ID | YFDY | NQKNY | ID NO: 27) | PLT (SEQ |
| | ID NO: 37) | NO: 38) | (SEQ ID | (SEQ ID | | ID NO: 41) |
| | | | NO: 39) | NO: 40) | | |
| 1-C09-1 | GYTFTSY | IDPSDSY | ARSPFDY | QNVGTN | SAS (SEQ | QQYNSYP |
| | W (SEQ ID | T (SEQ ID | (SEQ ID | (SEQ ID | ID NO: 43) | FT (SEQ |
| | NO: 32) | NO: 33) | NO: 34) | NO: 42) | | ID NO: 44) |
| 1-C09-3 | GYTFTSY | IDPSDSY | ARSPFDY | QNVGTN | SAS (SEQ | QQHNSYP |
| | W (SEQ ID | T (SEQ ID | (SEQ ID | (SEQ ID | ID NO: 43) | FT (SEQ |
| | NO: 32) | NO: 33) | NO: 34) | NO: 42) | | ID NO: 45) |
| 1-F09-1 | GYTFTSY | IDPSDSY | ARSPFDY | QDVSTA | SAS (SEQ | QQHNSYP |
| | W (SEQ ID | T (SEQ ID | (SEQ ID | (SEQ ID | ID NO: 43) | FT (SEQ |
| | NO: 32) | NO: 33) | NO: 34) | NO: 46) | | ID NO: 45) |
| 1-F12-1 | GYTFTSY | IDPSDSY | ARSPFDY | QDVSTA | SAS (SEQ | HQHNTY |
| | W (SEQ ID | T (SEQ ID | (SEQ ID | (SEQ ID | ID NO: 43) | PFT (SEQ |
| | NO: 32) | NO: 33) | NO: 34) | NO: 46) | | ID NO: 47) |
| 1-H01-1 | GYTFTSY | IDPSNSY | ARSPFDY | QNVGTN | SAS (SEQ | QQHNSYP |
| | W (SEQ ID | T (SEQ ID | (SEQ ID | (SEQ ID | ID NO: 43) | FT (SEQ |
| | NO: 32) | NO: 35) | NO: 34) | NO: 42) | | ID NO: 45) |
| 2-D11-1 | GYTFNTY | IDPSDSY | ARSPFDY | QNVGTN | SAS (SEQ | QQHNSYP |
| | W (SEQ ID | T (SEQ ID | (SEQ ID | (SEQ ID | ID NO: 43) | FT (SEQ |
| | NO: 36) | NO: 33) | NO: 34) | NO: 42) | | ID NO: 45) |

In some embodiments, provided herein is a PD-1 agonist antibody, wherein the antibody comprises the amino acid sequences of the following three VH CDRs: SEQ ID NO: 10, SEQ ID NO: 11, SEQ ID NO: 12; and/or comprises the amino acid sequences of the following three VL CDRs SEQ ID NO: 13, SEQ ID NO: 14, and SEQ ID NO: 15.

In some embodiments, provided herein is a PD-1 agonist antibody, wherein the antibody comprises the amino acid sequences of the following three VH CDRs: SEQ ID NO: 16, SEQ ID NO: 17, SEQ ID NO: 18; and/or comprises the amino acid sequences of the following three VL CDRs SEQ ID NO: 19, SEQ ID NO: 20, and SEQ ID NO: 21.

In some embodiments, provided herein is a PD-1 agonist antibody, wherein the antibody comprises the amino acid sequences of the following three VH CDRs: SEQ ID NO: 22, SEQ ID NO: 23, SEQ ID NO: 24; and/or comprises the amino acid sequences of the following three VL CDRs SEQ ID NO: 26, SEQ ID NO: 27, and SEQ ID NO: 28.

In some embodiments, provided herein is a PD-1 agonist antibody, wherein the antibody comprises the amino acid sequences of the following three VH CDRs: SEQ ID NO: 22, SEQ ID NO: 23, SEQ ID NO: 25; and/or comprises the amino acid sequences of the following three VL CDRs SEQ ID NO: 26, SEQ ID NO: 27, and SEQ ID NO: 28.

In some embodiments, provided herein is a PD-1 agonist antibody, wherein the antibody comprises the amino acid sequences of the following three VH CDRs: SEQ ID NO: 22, SEQ ID NO: 23, SEQ ID NO: 25; and/or comprises the amino acid sequences of the following three VL CDRs SEQ ID NO: 26, SEQ ID NO: 31, and SEQ ID NO: 28.

In some embodiments, provided herein is a PD-1 agonist antibody, wherein the antibody comprises the amino acid sequences of the following three VH CDRs: SEQ ID NO: 29, SEQ ID NO: 30, SEQ ID NO: 25; and/or comprises the amino acid sequences of the following three VL CDRs SEQ ID NO: 26, SEQ ID NO: 27, and SEQ ID NO: 28.

In some embodiments, provided herein is a PD-1 agonist antibody, wherein the antibody comprises the amino acid sequences of the following three VH CDRs: SEQ ID NO: 37, SEQ ID NO: 38, SEQ ID NO: 39; and/or comprises the amino acid sequences of the following three VL CDRs SEQ ID NO: 40, SEQ ID NO: 27, and SEQ ID NO: 41.

In some embodiments, provided herein is a PD-1 agonist antibody, wherein the antibody comprises the amino acid sequences of the following three VH CDRs: SEQ ID NO: 32, SEQ ID NO: 33, SEQ ID NO: 34; and/or comprises the amino acid sequences of the following three VL CDRs SEQ ID NO: 42, SEQ ID NO: 43, and SEQ ID NO: 44.

In some embodiments, provided herein is a PD-1 agonist antibody, wherein the antibody comprises the amino acid sequences of the following three VH CDRs: SEQ ID NO: 32, SEQ ID NO: 33, SEQ ID NO: 34; and/or comprises the amino acid sequences of the following three VL CDRs SEQ ID NO: 42, SEQ ID NO: 43, and SEQ ID NO: 45.

In some embodiments, provided herein is a PD-1 agonist antibody, wherein the antibody comprises the amino acid sequences of the following three VH CDRs: SEQ ID NO: 32, SEQ ID NO: 33, SEQ ID NO: 34; and/or comprises the amino acid sequences of the following three VL CDRs SEQ ID NO: 46, SEQ ID NO: 43, and SEQ ID NO: 45.

In some embodiments, provided herein is a PD-1 agonist antibody, wherein the antibody comprises the amino acid sequences of the following three VH CDRs: SEQ ID NO: 32, SEQ ID NO: 33, SEQ ID NO: 34; and/or comprises the amino acid sequences of the following three VL CDRs SEQ ID NO: 46, SEQ ID NO: 43, and SEQ ID NO: 47.

In some embodiments, provided herein is a PD-1 agonist antibody, wherein the antibody comprises the amino acid sequences of the following three VH CDRs: SEQ ID NO: 32, SEQ ID NO: 35, SEQ ID NO: 34; and/or comprises the amino acid sequences of the following three VL CDRs SEQ ID NO: 42, SEQ ID NO: 43, and SEQ ID NO: 45.

In some embodiments, provided herein is a PD-1 agonist antibody, wherein the antibody comprises the amino acid sequences of the following three VH CDRs: SEQ ID NO: 36, SEQ ID NO: 33, SEQ ID NO: 34; and/or comprises the amino acid sequences of the following three VL CDRs SEQ ID NO: 42, SEQ ID NO: 43, and SEQ ID NO: 45.

B. Exemplary PD-1 Agonist Antibodies—Variable Region Sequences.

The term variable domain and variable region are used interchangeably and refer to the portions of the light and heavy chains of an antibody that include the complementarity determining regions and framework regions (FRs).

Table 2 provides amino acid sequences for the variable domains of exemplary PD-1 agonist antibodies of the disclosure. Accordingly, in some embodiments a PD-1 agonist antibody of the disclosure comprises a variable heavy chain comprising an amino acid sequence selected from SEQ ID NOS: 1-5, 48-54; and/or in some embodiments a PD-1 agonist antibody of the disclosure comprises a variable light chain comprising an amino acid sequence selected from SEQ ID NOS: 6-9, 55-61.

In some embodiments, a PD-1 agonist antibody of the disclosure comprises the combination of VH/VL variable chain sequences of any one of the combinations listed in Table 2.

TABLE 2
Exemplary Variable Heavy Chain and Variable Light Chain Amino Acid
Sequences of PD-1 Agonist Antibodies.

| Antibody | Variable Heavy Chain | Variable Light Chain |
|---|---|---|
| ID | Amino Acid Sequence | Amino Acid Sequence |
| 7 | EVQLLESGGGLVQPGGSLRLSCA | DIQMTQSPSSLSASVGDRVTITCR |
| | ASGFPFGNYLMNWVRQAPGKG | ASETISNFLNWYQQKPGKAPKLL |
| | LEWVSVISGGGVSTYYADSVKG | IYTTSSLQRGVPSRFSGSGSGTDF |
| | RFTISRDNSKNTLYLQMNSLRAE | TLTISSLPEDFATYYCQQSYSVP |
| | DTAVYYCARGPSSWPAFDVWG | WTFGGGTKVEIK (SEQ ID NO: 6) |
| | QGTLVTVSS (SEQ ID NO: 1) | |
| 43 | EVQLLESGGGLVQPGGSLRLSCA | DIQMTQSPSSLSASVGDRVTITCR |
| | ASGFTFSIHPMTWVRQAPGKGLE | ASQSIDRFLNWYQQKPGKAPKL |
| | WVSLISTGGGSTYYADSVKGRFT | LIYGASNLPSGVPSRFSGSGSGTD |
| | ISRDNSKNTLYLQMNSLRAEDTA | FTLTISSLQPEDFATYYCQQSFSIP |
| | VYYCASASVLGSGGYWSPGMD | WTFGGGTKVEIK (SEQ ID NO: 7) |
| | YWGQGTLVTVSS (SEQ ID NO: 2) | |
| E2-B5- | EVQLVQSGAEVKKPGATVKISC | DIVMTQSPDSLAVSLGERATINC |
| 6_201 | KASGYSLSDHYMHWVQQAPGK | KSGQSVFYSSNNKNYLAWYQQ |
| | GLEWMGRIDPHSGATKYDPKFQ | KPGQPPKLLIYWASTRESGVPDR |
| | GRVTITADTSTDTAYMELSSLRS | FSGSGSGTDFTLTISSLQAEDVAV |
| | EDTAVYYCARSGPVHYYGSSYV | YYCQQYLYSWTFGQGTKLEIK |
| | MDYWGQGTTVTVSS (SEQ ID | (SEQ ID NO: 8) |
| | NO: 3) | |
| E2-B5- | EVQLVQSGAEVKKPGATVKISC | DIVMTQSPDSLAVSLGERATINC |
| 7_201 | KASGYSLSDHYMHWVQQAPGK | KSGQSVFYSSNNKNYLAWYQQ |
| | GLEWMGRIDPHSGATKYDPKFQ | KPGQPPKLLIYWASTRESGVPDR |
| | GRVTITADTSTDTAYMELSSLRS | FSGSGSGTDFTLTISSLQAEDVAV |
| | EDTAVYYCARSGPVYHYGSSYV | YYCQQYLYSWTFGQGTKLEIK |
| | MDYWGQGTTVTVSS (SEQ ID | (SEQ ID NO: 8) |
| | NO: 4) | |
| E2-B5- | EVQLVQSGAEVKKPGATVKISC | DIVMTQSPDSLAVSLGERATINC |
| 7_211 | KASGYSLSDHYMHWVQQAPGK | KSGQSVFYSSNNKNYLAWYQQ |
| | GLEWMGRIDPHSGATKYDPKFQ | KPGQPPKLLIYWADTRESGVPDR |
| | GRVTITADTSTDTAYMELSSLRS | FSGSGSGTDFTLTISSLQAEDVAV |
| | EDTAVYYCARSGPVYHYGSSYV | YYCQQYLYSWTFGQGTKLEIK |
| | MDYWGQGTTVTVSS (SEQ ID | (SEQ ID NO: 9) |
| | NO: 4) | |
| D4-A7- | EVQLVQSGAEVKKPGATVKISC | DIVMTQSPDSLAVSLGERATINC |
| 7_201 | KASGYILTDHYMHWVQQAPGK | KSGQSVFYSSNNKNYLAWYQQ |
| | GLEWMGRIDPHSGGIKYDPKFQ | KPGQPPKLLIYWASTRESGVPDR |
| | GRVTITADTSTDTAYMELSSLRS | FSGSGSGTDFTLTISSLQAEDVAV |
| | EDTAVYYCARSGPVYHYGSSYV | YYCQQYLYSWTFGQGTKLEIK |
| | MDYWGQGTTVTVSS (SEQ ID | (SEQ ID NO: 8) |
| | NO: 5) | |
| 27A5 | QVTLKESGPGILQPSQTLSLTCSF | DIVMTQSPSSLAVSVGEKVTLTC |
| | SGFSLSTFGMGVGWIRQPSGKGL | KSSQTLLDSTNQKNYLAWYQQI |
| | EWLAHIWWDADKYSTPALKSRL | PGQSPKLLIYWASTRESGVPDRF |
| | TISKDTSKNQVFLKIANVDTADT | TGSGSGTDFTLTISSVKAEDLTV |
| | ATYYCARIGRNYYFDYWGQGTT | YYCQQYYTYPLTFGAGTKLELK |
| | LTVSS (SEQ ID NO: 48) | (SEQ ID NO: 55) |
| 1-C09-1 | QVQLQQPGTELVRPGTSVKMSC | DIVMTQSQKFMSTSVGDRVSVT |
| | KASGYTFTSYWMHWVKQRPGQ | CKASQNVGTNVAWYQQKPGQS |
| | GLEWIGVIDPSDSYTQYNQKFKG | PKALIYSASYRYSGIPDRFTGSGS |
| | KATLTVDTSSSTAYMQLSSLTSE | GTDFTLTISNVQSEDLADFFCQQ |
| | DSAVYYCARSPFDYWGQGTTLT | YNSYPFTFGGGTKLEIK (SEQ ID |
| | VSS (SEQ ID NO: 49) | NO: 56) |
| 1-C09-3 | QVQLQQPGAELVRPGTSVKLSC | DIQMTQSQKFMSTSVGDRVSVT |
| | KASGYTFTSYWMHWVKERPGQ | CKASQNVGTNVAWYQQKPGQS |
| | GLEWIGVIDPSDSYTNYNQKFKD | PKALIYSASYRYSGVPDRFIGSGS |
| | KATLTVDTSSSTAYMQLSSLTSE | GTDFTLTIRNVQSEDLAEYFCQQ |
| | DSAVYYCARSPFDYWGQGTTLT | HNSYPFTFGGGTKLEIK (SEQ ID |
| | VSS (SEQ ID NO: 50) | NO: 57) |
| 1-F09-1 | QVQLQQPGAEVVMPGASVKLSC | DIQMNQSHKFMSTSVGDRVSITC |
| | KASGYTFTSYWMHWVKQRPGQ | KASQDVSTAVAWYQQKPGQSP |
| | GLEWIGVIDPSDSYTQYNQKFKG | KALIYSASNRYSGVPDRFIGSGS |
| | KATLTVDTSSSTAYMQLSSLTSE | GTDFTLTIRNVQSEDLAEYFCQQ |
| | DSAVYYCARSPFDYWGQGTTLT | HNSYPFTFGGGTKLEMK (SEQ ID |
| | VSS (SEQ ID NO: 51) | NO: 58) |
| 1-F12-1 | EVQLQQPGAELVRPGTSVKLSC | DIVMTQSHKFMSTSVGDRVSITC |
| | KASGYTFTSYWMHWVKQRPGQ | KASQDVSTAVAWYQQKPGQSP |
| | GLEWIGVIDPSDSYTQYNQKFKG | KALIYSASYRYSGVPDRFTGSGS |
| | KATLTVDTSSSTAYMQLSSLTSE | GTDFTLTISNVQAEDLAVYYCH |
| | DSAVYYCARSPFDYWGQGTTVT | QHNTYPFTFGGGTKLEIK (SEQ |
| | VSS (SEQ ID NO: 52) | ID NO: 59) |
| 1-H01-1 | QVQLQQPGAELVKPGASVKMSC | DVVMTQSQKFMSTSVGDRVSVT |
| | KASGYTFTSYWMHWVKQRPGQ | CKASQNVGTNVAWYQQKPGQS |
| | GLEWIGVIDPSNSYTIYNQKFKG | PKALIYSASYRYSGVPDRFTGSG |
| | KATLTVDTSSSTAYMQLSSLTSE | SGTDFTLTIRNVQSEDLAEYFCQ |
| | DSAVYYCARSPFDYWGQGTTLT | QHNSYPFTFGGGTKLEIK (SEQ |
| | VSS (SEQ ID NO: 53) | ID NO: 60) |
| 2-D11-1 | EVQLQQPGAELVRPGTSVKLAC | DVQMTQSQKFMSTSVGDRVSVT |
| | KASGYTFNTYWMHWVNQRPGQ | CKASQNVGTNVAWYQQKPGQS |
| | GLEWIGVIDPSDSYTQYNQKFKG | PKALIYSASYRYSGVPDRFIGSGS |
| | KATLTVDTSSSTAYMQLSSLTSE | GTDFTLTISNVQSEDLAEYFCQQ |
| | DSAVYYCARSPFDYWGQGTTLT | HNSYPFTFGGGTKLEIK (SEQ ID |
| | VSS (SEQ ID NO: 54) | NO: 61) |

TABLE 3
Exemplary Variable Heavy Chain and Variable Light Chain Nucleic
Acid Sequences of PD-1 Agonist Antibodies.

| Antibody | Variable Heavy Chain | Variable Light Chain |
|---|---|---|
| ID | Nucleic Acid Sequence | Nucleic Acid Sequence |
| 7 | GAGGTGCAGCTGCTGGAGTCC | GACATCCAGATGACCCAGTCCC |
| | GGCGGCGGCCTGGTGCAGCCC | CTCCTCCCTGTCCGCCTCCGTG |
| | GGCGGCTCCCTGAGGCTGTCCT | GGCGACAGGGTGACCATCACC |
| | GCGCCGCCTCCGGCTTCCCCTT | TGCAGGGCCTCCGAGACCATCT |
| | CGGCAACTACCTGATGAACTGG | CCAACTTCCTGAACTGGTACCA |
| | GTGAGGCAGGCCCCCGGCAAG | GCAGAAGCCCGGCAAGGCCCC |
| | GGCCTGGAGTGGGTGTCCGTGA | CAAGCTGCTGATCTACACCACC |
| | TCTCCGGCGGCGGCGTGTCCAC | TCCTCCCTGCAGAGGGGCGTGC |
| | CTACTACGCCGACTCCGTGAAG | CCTCCAGGTTCTCCGGCTCCGG |
| | GGCAGGTTCACCATCTCCAGGG | CTCCGGCACCGACTTCACCCTG |
| | ACAACTCCAAGAACACCCTGTA | ACCATCTCCTCCCTGCAGCCCG |
| | CCTGCAGATGAACTCCCTGAGG | AGGACTTCGCCACCTACTACTG |
| | GCCGAGGACACCGCCGTGTACT | CCAGCAGTCCTACTCCGTGCCC |
| | ACTGCGCCAGGGGCCCCTCCTC | TGGACCTTCGGCGGCGGCACCA |
| | CTGGCCCGCCTTCGACGTGTGG | AGGTGGAGATCAAG (SEQ ID |
| | GGCCAGGGCACCCCTGGTGAC | NO: 67) |
| | CGTGTCCTCC (SEQ ID NO: 66) | |
| 43 | GAGGTGCAGCTGCTGGAGTCC | GACATCCAGATGACCCAGTCCC |
| | GGCGGCGGCCTGGTGCAGCCC | CTCCTCCCTGTCCGCCTCCGTG |
| | GGCGGCTCCCTGAGGCTGTCCT | GGCGACAGGGTGACCATCACC |
| | GCGCCGCCTCCGGCTTCACCTT | TGCAGGGCCTCCCAGTCCATCG |
| | CTCCATCCACCCCATGACCTGG | ACAGGTTCCTGAACTGGTACCA |
| | GTGAGGCAGGCCCCCGGCAAG | GCAGAAGCCCGGCAAGGCCCC |
| | GGCCTGGAGTGGGTGTCCCTGA | CAAGCTGCTGATCTACGGCGCC |
| | TCTCCACCGGCGGCGGCTCCAC | TCCAACCTGCCCTCCGGCGTGC |
| | CTACTACGCCGACTCCGTGAAG | CCTCCAGGTTCTCCGGCTCCGG |
| | GGCAGGTTCACCATCTCCAGGG | CTCCGGCACCGACTTCACCCTG |
| | ACAACTCCAAGAACACCCTGTA | ACCATCTCCTCCCTGCAGCCCG |
| | CCTGCAGATGAACTCCCTGAGG | AGGACTTCGCCACCTACTACTG |
| | GCCGAGGACACCGCCGTGTACT | CCAGCAGTCCTTCTCCATCCCC |
| | ACTGCGCCTCCGCCTCCGTGCT | TGGACCTTCGGCGGCGGCACCA |
| | GGGCTCCGGCGGCTACTGGTCC | AGGTGGAGATCAAG (SEQ ID |
| | CCCGGCATGGACTACTGGGGCC | NO: 69) |
| | AGGGCACCCCTGGTGACCGTGT | |
| | CCTCC (SEQ ID NO: 68) | |
| E2-B5- | GAGGTGCAGCTGGTGCGTCCG | GACATCGTGATGACCCAGTCCC |
| 6_201 | GCGCCGAGGTGAAGAAGCCCG | CCGACTCCCTGGCCGTGTCCCT |
| | GCGCCACCGTGAAGATCTCCTG | GGGCGAGAGGGCCACCATCAA |
| | CAAGGCCTCCGGCTACTCCCTG | CTGCAAGTCCGGCCAGTCCGTG |
| | TCCGACCACTACATGCACTGGG | TTCTACTCCTCCACAACAAGAA |
| | TGCAGCAGGCCCCCGGCAAGG | CTACCTGGCCTGGTACCAGCAG |
| | GCCTGGAGTGGATGGGCAGGA | AAGCCCGGCCAGCCCCCCAAG |
| | TCGACCCCCACTCCGGCGCCAC | CTGCTGATCTACTGGGCCTCCA |
| | CAAGTACGACCCCAAGTTCCAG | CCAGGAGTCCGGCGTGTCCCGA |
| | GGCAGGGTGACCATCACCGCC | CAGGTTCTCCGGCTCCGGCTCC |
| | GACACCTCCACCGACACCGCCT | GGCACCGACTTCACCCTGACCA |
| | ACATGGAGCTGTCCTCCCTGAG | TCTCCTCCCTGCAGGCCGAGGA |
| | GTCCGAGGACACCGCCGTGTAC | CGTGGCCGTGTACTACTGCCAG |
| | TACTGCGCCAGGTCCGGCCCCC | CAGTACCTGTACTCCTGGACCT |
| | GTCACTACTACGGCTCCTCCTA | TCGGCCAGGGCACCAAGCTGG |
| | CGTGATGGACTACTGGGGCCA | AGATCAAG (SEQ ID NO: 71) |
| | GGGCACCAACCGTGACCGTGTC | |
| | CTCC (SEQ ID NO: 70) | |
| E2-B5- | GAGGTGCAGCTGGTGCGTCCG | GACATCGTGATGACCCAGTCCC |
| 7_201 | GCGCCGAGGTGAAGAAGCCCG | CCGACTCCCTGGCCGTGTCCCT |
| | GCGCCACCGTGAAGATCTCCTG | GGGCGAGAGGGCCACCATCAA |
| | CAAGGCCTCCGGCTACTCCCTG | CTGCAAGTCCGGCCAGTCCGTG |
| | TCCGACCACTACATGCACTGGG | TTCTACTCCTCCACAACAAGAA |
| | TGCAGCAGGCCCCCGGCAAGG | CTACCTGGCCTGGTACCAGCAG |
| | GCCTGGAGTGGATGGGCAGGA | AAGCCCGGCCAGCCCCCCAAG |
| | TCGACCCCCACTCCGGCGCCAC | CTGCTGATCTACTGGGCCTCCA |
| | CAAGTACGACCCCAAGTTCCAG | CCAGGAGTCCGGCGTGTCCCGA |
| | GGCAGGGTGACCATCACCGCC | CAGGTTCTCCGGCTCCGGCTCC |
| | GACACCTCCACCGACACCGCCT | GGCACCGACTTCACCCTGACCA |
| | ACATGGAGCTGTCCTCCCTGAG | TCTCCTCCCTGCAGGCCGAGGA |
| | GTCCGAGGACACCGCCGTGTAC | CGTGGCCGTGTACTACTGCCAG |
| | TACTGCGCCAGGTCCGGCCCCC | CAGTACCTGTACTCCTGGACCT |
| | GTGTACCACTACGGCTCCTCCT | TCGGCCAGGGCACCAAGCTGG |
| | ACGTGATGGACTACTGGGGCC | AGATCAAG (SEQ ID NO: 73) |
| | AGGGCACCAACCGTGACCGTG | |
| | TCCTCC (SEQ ID NO: 72) | |
| E2-B5- | GAGGTGCAGCTGGTGCGTCCG | GACATCGTGATGACCCAGTCCC |
| 7_211 | GCGCCGAGGTGAAGAAGCCCG | CCGACTCCCTGGCCGTGTCCCT |
| | GCGCCACCGTGAAGATCTCCTG | GGGCGAGAGGGCCACCATCAA |
| | CAAGGCCTCCGGCTACTCCCTG | CTGCAAGTCCGGCCAGTCCGTG |
| | TCCGACCACTACATGCACTGGG | TTCTACTCCTCCACAACAAGAA |
| | TGCAGCAGGCCCCCGGCAAGG | CTACCTGGCCTGGTACCAGCAG |
| | GCCTGGAGTGGATGGGCAGGA | AAGCCCGGCCAGCCCCCCAAG |
| | TCGACCCCCACTCCGGCGCCAC | CTGCTGATCTACTGGGCCGACA |
| | CAAGTACGACCCCAAGTTCCAG | CCAGGAGTCCGGCGTGTCCCGA |
| | GGCAGGGTGACCATCACCGCC | CAGGTTCTCCGGCTCCGGCTCC |
| | GACACCTCCACCGACACCGCCT | GGCACCGACTTCACCCTGACCA |
| | ACATGGAGCTGTCCTCCCTGAG | TCTCCTCCCTGCAGGCCGAGGA |
| | GTCCGAGGACACCGCCGTGTAC | CGTGGCCGTGTACTACTGCCAG |
| | TACTGCGCCAGGTCCGGCCCCC | CAGTACCTGTACTCCTGGACCT |
| | GTGTACCACTACGGCTCCTCCT | TCGGCCAGGGCACCAAGCTGG |
| | ACGTGATGGACTACTGGGGCC | AGATCAAG (SEQ ID NO: 74) |
| | AGGGCACCAACCGTGACCGTG | |
| | TCCTCC (SEQ ID NO: 74) | |
| D4-A7- | GAGGTGCAGCTGGTGCGTCCG | GACATCGTGATGACCCAGTCCC |
| 7_201 | GCGCCGAGGTGAAGAAGCCCG | CCGACTCCCTGGCCGTGTCCCT |
| | GCGCCACCGTGAAGATCTCCTG | GGGCGAGAGGGCCACCATCAA |
| | CAAGGCCTCCGGCTACATCCTG | CTGCAAGTCCGGCCAGTCCGTG |
| | ACCGACCACTACATGCACTGGG | TTCTACTCCTCCACAACAAGAA |
| | TGCAGCAGGCCCCCGGCAAGG | CTACCTGGCCTGGTACCAGCAG |
| | GCCTGGAGTGGATGGGCAGGA | AAGCCCGGCCAGCCCCCCAAG |
| | TCGACCCCCACTCCGGCGGCAT | CTGCTGATCTACTGGGCCTCCA |
| | CAAGTACGACCCCAAGTTCCAG | CCAGGGAGTCCGGCGTGTCCCG |
| | GGCAGGGTGACCATCACCGCC | ACAGGTTCTCCGGCTCCGGCTC |
| | GACACCTCCACCGACACCGCCT | CGGCACCGACTTCACCCTGACC |
| | ACATGGAGCTGTCCTCCCTGAG | ATCTCCTCCCTGCAGGCCGAGG |
| | GTCCGAGGACACCGCCGTGTAC | ACGTGGCCGTGTACTACTGCCA |
| | TACTGCGCCAGGTCCGGCCCCC | GCAGTACCTGTACTCCTGGACC |
| | GTGTACCACTACGGCTCCTCCT | TTCGGCCAGGGCACCAAGCTG |
| | ACGTGATGGACTACTGGGGCC | GAGATCAAG (SEQ ID NO: 77) |
| | AGGGCACCAACCGTGACCGTG | |
| | TCCTCC (SEQ ID NO: 76) | |
| 27A5 | CAGGTGACCCTGAAGGAGTCC | GACATCGTGATGACCCAGTCCC |
| | GGCCCCGGCATCCTGCAGCCCT | CCTCCTCCCTGGCCGTGTCCGT |
| | CCCAGACCCTGTCCCTGACCTG | GGGCGAGAAGGTGACCCTGAC |
| | CTCCTTCTCCGGCTTCTCCCTGT | CTGCAAGTCCTCCCAGACCCTG |
| | CCACCTTCGGCATGGGCGTGGG | CTGGACTCCACCAACCAGAAG |
| | CTGGATCAGGCAGCCCTCCGGC | AACTACCTGGCCTGGTACCAGC |
| | AAGGGCCTGGAGTGGCTGGCC | AGATCCCCGGCCAGTCCCCCCA |
| | CACATCTGGTGGGACGCCGAC | AGCTGCTGATCTACTGGGCCTC |
| | AAGTACTCCACCCCCGCCCTGA | CACCAGGGAGTCCGGCGTGCC |
| | AGTCCAGGCTGACCATCTCCAA | CGACAGGTTCACCGGCTCCGGC |
| | GGACACCTCCAAGAACCAAGT | TCCGGCACCGACTTCACCCTGA |
| | GTTCCTGAAGATCGCCAACGTG | CCATCTCCTCCGTGAAGGCCGA |
| | GACACCGCCGACACCGCCACCT | GGACCTGACCGTGTACTACTGC |
| | ACTACTGCGCCAGGATCGGCA | CAGCAGTACTACACCTACCCCC |
| | GGAACTACTACTTCGACTACTG | TGACCTTCGGCGCCGGCACCAA |
| | GGGCCAGGGCACCAACCCTGA | GCTGGAGCTGAAG (SEQ ID NO: |
| | CCGTGTCCTCC (SEQ ID NO: 78) | 79) |
| 1-C09-1 | CAGGTGCAGCTGCAGCAGCCC | GACATCGTGATGACCCAGTCCC |
| | GGCACCGAGCTGGTGAGGCCCg | AGAAGTTCATGTCCACCTCCGT |
| | gCACCTCCGTGAAGATGTCCTG | GGGCGACAGGGTGTCCGTGAC |
| | CAAGGCCTCCGGCTACACCTTC | CTGCAAGGCCTCCCAGAACGTG |
| | ACCTCCTACTGGATGCACTGGG | GGCACCAACGTGGCCTGGTACC |
| | TGAAGCAGAGGCCCggCCAGGG | AGCAGAAGCCCGGCCAGTCCC |
| | CCTGGAGTGGATCGGCGTGATC | CCAAGGCCCTGATCTACTCCGC |
| | GACCCCTCCGACTCCTACACCC | CTCCTACAGGTACTCCGGCATC |
| | AGTACAACCAGAAGTTCAAGG | CCCGACAGGTTCACCGGCTCCG |
| | GCAAGGCCACCCTGACCGTGG | GCTCCGGCACCGACTTCACCCT |
| | ACACCTCCTCCTCCACCGCCTA | GACCATCTCCAACGTGCAGTCC |
| | CATGCAGCTGTCCTCCCTGACC | GAGGACCTGGCCGACTTCTTCT |
| | TCCGAGGACTCCGCCGTGTACT | GCCAGCAGTACAACTCCTACCC |
| | ACTGCCCGAGTCCCCCTTCGAC | CTTCACCTTCGGCGGCGGCACC |
| | TACTGGGGCCAGGGCACCAAC | AAGCTGGAGATCAAG (SEQ ID |
| | CCTGACCGTGTCCTCC (SEQ ID | NO: 81) |
| | NO: 80) | |
| 1-C09-3 | CAGGTGCAGCTGCAGCAGCCC | GACATCCAGATGACCCAGTCCC |
| | GGCGCCGAGCTGGTGAGGCCCg | AGAAGTTCATGTCCACCTCCGT |
| | gCACCTCCGTGAAGCTGTCCTG | GGGCGACAGGGTGTCCGTGAC |
| | CAAGGCCTCCGGCTACACCTTC | CTGCAAGGCCTCCCAGAACGTG |
| | ACCTCCTACTGGATGCACTGGG | GGCACCAACGTGGCCTGGTACC |
| | TGAAGGAGAGGCCCggCCAGGG | AGCAGAAGCCCGGCCAGTCCC |
| | CCTGGAGTGGATCGGCGTGATC | CCAAGGCCCTGATCTACTCCGC |
| | GACCCCTCCGACTCCTACACCA | CTCCTACAGGTACTCCGGCGTG |
| | ACTACAACCAGAAGTTCAAGG | CCCGACAGGTTCATCGGCTCCG |
| | ACAAGGCCACCCTGACCGTGG | GCTCCGGCACCGACTTCACCCT |
| | ACACCTCCTCCTCCACCGCCTA | GACCATCAGGAACGTGCAGTC |
| | CATGCAGCTGTTCCTCCCTGAC | CGAGGACCTGGCCGAGTACTTC |
| | CTCCGAGGACTCCGCCGTGTAC | TGCCAGCAGCACAACCTCCTAC |
| | TACTGCCAGGTCCCCCTTCGAC | CCCTTCACCTTCGGCGGCGGCA |
| | TACTGGGGCCAGGGCACCAAC | CCAAGCTGGAGATCAAG (SEQ |
| | CCTGACCGTGTCCTCC (SEQ ID | ID NO: 83) |
| | NO: 82) | |
| 1-F09-1 | CAGGTGCAGCTGCAGCAGCCC | GACATCCAGATGAACCAGTCCC |
| | GGCGCCGAGGTGGTGATGCCC | ACAAGTTCATGTCCACCTCCGT |
| | GGCGCCTCCGTGAAGCTGTCCT | GGGCGACAGGGTGTCCATCAC |
| | GCAAGGCCTCCGGCTACACCTT | CTGCAAGGCCTCCCAGGACGTG |
| | CACCTCCTACTGGATGCACTGG | TCCACCGCCGTGGCCTGGTACC |
| | GTGAAGCAGAGGCCCAGCCAG | AGCAGAAGCCCGGCCAGTCCC |
| | GGCCTGGAGTGGATCGGCGTG | CCAAGGCCCCTGATCTACTCCG |
| | ATCGACCCCTCCGACTCCTACA | CCTCCAACAGGTACTCCGGCGT |
| | CCCAGTACAACCAGAAGTTCA | GCCCGACAGGTTCATCGGCTCC |
| | AGGGCAAGGCCACCCTGACCG | GGCTCCGGCACCGACTTCACCC |
| | TGGACACCTCCTCCTCCACCGC | TGACCATCAGGAACGTGCAGTC |
| | CTACATGCAGCTGTCCTCCCTG | CGAGGACCTGGCCGAGTACTTC |
| | ACCTCCGAGGACTCCGCCGTGT | TGCCAGCAGCACAACCTCACCC |
| | ACTACTGCGCCAGGTCCCCCTT | CTTCACCTTCGGCGGCGGCACC |
| | CGACTACTGGGGCCAGGGCAC | AAGCTGGAGATGAAG (SEQ ID |
| | CAACCCTGACCGTGTCCTCC | NO: 85) |
| | (SEQ ID NO: 84) | |
| 1-F12-1 | GAGGTGCAGCTGCAGCAGCCC | GACATCGTGATGACCCAGTCCC |
| | GGCGCCGAGCTGGTGAGGCCC | ACAAGTTCATGTCCACCTCCGT |
| | GGCACCTCCGTGAAGCTGTCCT | GGGCGACAGGGTGTCCATCAC |
| | GCAAGGCCTCCGGCTACACCTT | CTGCAAGGCCTCCCAGGACGTG |
| | CACCTCCTACTGGATGCACTGG | TCCACCGCCGTGGCCTGGTACC |
| | GTGAAGCAGAGGCCCGGCCAG | AGCAGAAGCCCGGCCAGTCCC |
| | GGCCTGGAGTGGATCGGCGTG | CCAAGGCCCTGATCTACTCCGC |
| | ATCGACCCCTCCGACTCCTACA | CTCCTACAGGTACTCCGGCGTG |
| | CCCAGTACAACCAGAAGTTCA | CCCGACAGGTTCACCGGCTCCG |
| | AGGGCAAGGCCACCCTGACCG | GCTCCGGCACCGACTTCACCCT |
| | TGGACACCTCCTCCTCCACCGC | GACCATCTCCAACGTGCAGGCC |
| | CTACATGCAGCTGTCCTCCCTG | GAGGACCTGGCCGTGTACTACT |
| | ACCTCCGAGGACTCCGCCGTGT | GCCACCAGCACAACACCTACCC |
| | ACTACTGCGCCAGGTCCCCCTT | CTTCACCTTCGGCGGCGGCACC |
| | CGACTACTGGGGCCAGGGCAC | AAGCTGGAGATCAAG (SEQ ID |
| | CACCGTGACCGTGTCCTCC | NO: 87) |
| | (SEQ ID NO: 86) | |
| 1-H01-1 | CAGGTGCAGCTGCAGCAGCCC | GACGTGGTGATGACCCAGTCCC |
| | GGCGCCGAGCTGGTGAAGCCC | AGAAGTTCATGTCCACCTCCGT |
| | GGCGCCTCCGTGAAGATGTCCT | GGGCGACAGGGTGTCCGTGAC |
| | GCAAGGCCTCCGGCTACACCTT | CTGCAAGGCCTCCCAGAACGTG |
| | CACCTCCTACTGGATGCACTGG | GGCACCAACGTGGCCTGGTACC |
| | GTGAAGCAGAGGCCCAGCCAG | AGCAGAAGCCCGGCCAGTCCC |
| | GGCCTGGAGTGGATCGGCGTG | CCAAGGCCCTGATCTACTCCGC |
| | ATCGACCCCTCCAACTCCTACA | CTCCTACAGGTACTCCGGCGTG |
| | CCATCTACAACCAGAAGTTCAA | CCCGACAGGTTCACCGGCTCCG |
| | GGGCAAGGCCACCCTGACCCT | GCTCCGGCACCGACTTCACCCT |
| | GGACACCTCCTCCTCCACCGCC | GACCATCAGGAACGTGCAGTC |
| | TACATGCAGCTGTGCTCCCTGA | CGAGGACCTGGCCGAGTACTTC |
| | CCTCCGAGGACTCCGCCGTGTA | TGCCAGCAGCACAACCTCCTAC |
| | CTACTGCCAGGTCCCCCTTCGA | CCCTTCACCTTCGGCGGCGGCA |
| | CTACTGGGGCCAGGGCACCAA | CCAAGCTGGAGATCAAG (SEQ |
| | CCTGACCGTGTGCTCC (SEQ ID | ID NO: 89) |
| | NO: 88) | |
| 2-D11-1 | GAGGTGCAGCTGCAGCAGCCC | GACGTGCAGATGACCCAGTCCC |
| | GGCGCCGAGCTGGTGAGGCCC | AGAAGTTCATGTCCACCTCCGT |
| | GGCACCTCCGTGAAGCTGGCCT | GGGCGACAGGGTGTCCGTGAC |
| | GCAAGGCCTCCGGCTACACCTT | CTGCAAGGCCTCCCAGAACGTG |
| | CAACACCTACTGGATGCACTGG | GGCACCAACGTGGCCTGGTACC |
| | GTGAACCAGAGGCCCGGCCAG | AGCAGAAGCCCGGCCAGTCCC |
| | GGCCTGGAGTGGATCGGCGTG | CCAAGGCCCTGATCTACTCCGC |
| | ATCGACCCCTCCGACTCCTACA | CTCCTACAGGTACTCCGGCGTG |
| | CCCAGTACAACCAGAAGTTCA | CCCGACAGGTTCATCGGCTCCG |
| | AGGGCAAGGCCACCCTGACCG | GCTCCGGCACCGACTTCACCCT |
| | TGGACACCTCCTCCTCCACCGC | GACCATCTCCAACGTGCAGTCC |
| | CTACATGCAGCTGTCCTCCCTG | GAGGACCTGGCCGAGTACTTCT |
| | ACCTCCGAGGACTCCGCCGTGT | GCCAGCAGCACAACTCCTACCC |
| | ACTACTGCGCCAGGTCCCCCTT | CTTCACCTTCGGCGGCGGCACC |
| | CGACTACTGGGGCCAGGGCAC | AAGCTGGAGATCAAG (SEQ ID |
| | CACCCTGACCGTGTCCTCC | NO: 91) |
| | (SEQ ID NO: 90) | |

In some embodiments, provided herein is a PD-1 agonist antibody, wherein the heavy chain variable domain (VH) of the antibody comprises the amino acid sequence of SEQ ID NO: 1; and/or wherein the light chain variable domain (VL) of the antibody comprises the amino acid sequence of SEQ ID NO: 6.

In some embodiments, provided herein is a PD-1 agonist antibody, wherein the heavy chain variable domain (VH) of the antibody comprises the amino acid sequence of SEQ ID NO: 2; and/or wherein the light chain variable domain (VL) of the antibody comprises the amino acid sequence of SEQ ID NO: 7.

In some embodiments, provided herein is a PD-1 agonist antibody, wherein the heavy

chain variable domain (VH) of the antibody comprises the amino acid sequence of SEQ ID NO: 3; and/or wherein the light chain variable domain (VL) of the antibody comprises the amino acid sequence of SEQ ID NO: 8.

In some embodiments, provided herein is a PD-1 agonist antibody, wherein the heavy chain variable domain (VH) of the antibody comprises the amino acid sequence of SEQ ID NO: 4; and/or wherein the light chain variable domain (VL) of the antibody comprises the amino acid sequence of SEQ ID NO: 8.

In some embodiments, provided herein is a PD-1 agonist antibody, wherein the heavy chain variable domain (VH) of the antibody comprises the amino acid sequence of SEQ ID NO: 4; and/or wherein the light chain variable domain (VL) of the antibody comprises the amino acid sequence of SEQ ID NO: 9.

In some embodiments, provided herein is a PD-1 agonist antibody, wherein the heavy chain variable domain (VH) of the antibody comprises the amino acid sequence of SEQ ID NO: 5; and/or wherein the light chain variable domain (VL) of the antibody comprises the amino acid sequence of SEQ ID NO: 8.

In some embodiments, provided herein is a PD-1 agonist antibody, wherein the heavy chain variable domain (VH) of the antibody comprises the amino acid sequence of SEQ ID NO: 48; and/or wherein the light chain variable domain (VL) of the antibody comprises the amino acid sequence of SEQ ID NO: 55.

In some embodiments, provided herein is a PD-1 agonist antibody, wherein the heavy chain variable domain (VH) of the antibody comprises the amino acid sequence of SEQ ID NO: 49; and/or wherein the light chain variable domain (VL) of the antibody comprises the amino acid sequence of SEQ ID NO: 56.

In some embodiments, provided herein is a PD-1 agonist antibody, wherein the heavy

chain variable domain (VH) of the antibody comprises the amino acid sequence of SEQ ID NO: 50; and/or wherein the light chain variable domain (VL) of the antibody comprises the amino acid sequence of SEQ ID NO: 57.

In some embodiments, provided herein is a PD-1 agonist antibody, wherein the heavy chain variable domain (VH) of the antibody comprises the amino acid sequence of SEQ ID NO: 51; and/or wherein the light chain variable domain (VL) of the antibody comprises the amino acid sequence of SEQ ID NO: 58.

In some embodiments, provided herein is a PD-1 agonist antibody, wherein the heavy chain variable domain (VH) of the antibody comprises the amino acid sequence of SEQ ID NO: 52; and/or wherein the light chain variable domain (VL) of the antibody comprises the amino acid sequence of SEQ ID NO: 59.

In some embodiments, provided herein is a PD-1 agonist antibody, wherein the heavy chain variable domain (VH) of the antibody comprises the amino acid sequence of SEQ ID NO: 53; and/or wherein the light chain variable domain (VL) of the antibody comprises the amino acid sequence of SEQ ID NO: 60.

In some embodiments, provided herein is a PD-1 agonist antibody, wherein the heavy chain variable domain (VH) of the antibody comprises the amino acid sequence of SEQ ID NO: 54; and/or wherein the light chain variable domain (VL) of the antibody comprises the amino acid sequence of SEQ ID NO: 61.

C. Exemplary Tandem scFv-Fc PD-1 Agonist Antibodies.

In some embodiments, the disclosure provides for tandem scFv antibodies, with multiple PD-1 binding sites. Tandem scFv-Fc antibodies of the disclosure are composed two or more scFv binding sites in tandem on each antibody arm, optionally linked by a linker, optionally a flexible linker. In some embodiments, a tandem scFV antibody has a total of four or more scFv binding sites in a single scFv-Fc formatted antibody. FIG. 11 illustrates an exemplary tandem scFv-Fc formatted antibody structure of the disclosure.

Without being held to theory or mechanism, PD-1 agonism is believed to be driven by clustering of multiple PD-1 receptors, and thus a tandem scFv-Fc antibody with multiple PD-1 binding sites may exhibit stronger PD-1 agonism when compared to the analogous traditional two binding site antibody, or even an scFv with a single VH and single VL.

More specifically, an exemplary tandem scFv-Fc PD-1 agonist antibody comprises two antibody arms, and an scFv1 and an scFv2 binding site arranged in tandem on each antibody arm and wherein said scFv1 and scFv2 are linked by a linker, optionally a flexible linker. FIG. 11 illustrates such an exemplary tandem scFv-Fc formatted antibody structure of the disclosure. Such antibody has a total of four scFv binding sites in a single tandem scFv-Fc formatted antibody.

In some embodiments, the scFv1 of each antibody arm comprises a first heavy chain variable domain (VH1) and a first light chain variable domain (VL1); and the scFv2 of each antibody arm comprises a first heavy chain variable domain (VH2) and a first light chain variable domain (VL2).

In some embodiments, the VH1 region comprises the amino acid sequence of any one of SEQ ID NOS: 1-5 and 48-54, more preferably SEQ ID NO: 1 or 53; and the VL1 region comprises the amino acid sequence of any one of SEQ ID NOS: 6-9 and 55-61, or preferably SEQ ID NO: 6 or 60, giving rise to scFv1. Likewise, in some embodiments, the VH2 region comprises the amino acid sequence of any one of SEQ ID NOS: 1-5 and 48-54, more preferably SEQ ID NO: 1 or 53; and the VL2 region comprises the amino acid sequence of any one of SEQ ID NOS: 6-9 and 55-61, or preferably SEQ ID NO: 6 or 60 giving rise to scFv2.

The VH1 and the VL1 of each scFV1 may be connected by a linker, e.g. a flexible linker.

The VH2 and the VL2 of each scFV2 may be connected by a linker, e.g. a flexible linker.

The scFvs on each antibody arm may be connected by a linker, e.g. a flexible linker. An exemplary linker comprises the following amino acid sequence: GGGGSGGGGSGGGGS (SEQ ID NO: 64).

TABLE 4
Exemplary Tandem scFv-Fc Amino Acid Sequences.

| | Variable | Variable | | Variable | Variable |
|---|---|---|---|---|---|
| | Heavy Chain | Light Chain | | Heavy Chain | Light Chain |
| | Amino Acid | Amino Acid | scFv1- | Amino Acid | Amino Acid |
| Antibody | Sequence 1 | Sequence 1 | scFv2 | Sequence 2 | Sequence 2 |
| ID | (VH1) | (VL1) | Linker | (VH2) | (VL2) |
| Tandem 7 | EVQLLESGG | DIQMTQSPSS | GGGG | EVQLLESGGG | DIQMTQSP |
| monospecific | GLVQPGGSL | LSASVGDRV | SGGG | LVQPGGSLRL | SSLSASVG |
| | RLSCAASGFP | TITCRASETIS | GSGG | SCAASGFPFG | DRVTITCR |
| | FGNYLMNW | NFLNWYQQ | GGS | NYLMNWVR | ASETISNFL |
| | VRQAPGKGL | KPGKAPKLLI | (SEQ ID | QAPGKGLEW | NWYQQKP |
| | EWVSVISGG | YTTSSLQRG | NO: | VSVISGGGVS | GKAPKLLI |
| | GVSTYYADS | VPSRFSGSGS | 64) | TYYADSVKG | YTTSSLQR |
| | VKGRFTISRD | GTDFTLTISS | | RFTISRDNSK | GVPSRFSG |
| | NSKNTLYLQ | LQPEDFATY | | NTLYLQMNS | SGSGTDFT |
| | MNSLRAEDT | YCQQSYSVP | | LRAEDTAVY | LTISSLQPE |
| | AVYYCARGP | WTFGGGTKV | | YCARGPSSW | DFATYYCQ |
| | SSWPAFDVW | EIK (SEQ ID | | PAFDVWGQG | QSYSVPWT |
| | GQGTLVTVSS | NO: 6) | | TLVTVSS | FGGGTKVE |
| | (SEQ ID NO: | | | (SEQ ID NO: 1) | IK (SEQ ID |
| | 1) | | | | NO: 6) |
| Tandem | QVQLQQPGA | DVVMTQSQK | | QVQLQQPGA | DVVMTQS |
| 1-H01-1 | ELVKPGASV | FMSTSVGDR | GGGG | ELVKPGASV | QKFMSTSV |
| monospecific | KMSCKASGY | VSVTCKASQ | SGGG | KMSCKASGY | GDRVSVTC |
| | TFTSYWMHW | NVGTNVAW | GSGG | TFTSYWMHW | KASQNVGT |
| | VKQRPGQGL | YQQKPGQSP | GGS | VKQRPGQGL | NVAWYQQ |
| | EWIGVIDPSN | KALIYSASYR | (SEQ | EWIGVIDPSN | KPGQSPKA |
| | SYTIYNQKFK | YSGVPDRFT | ID NO: | SYTIYNQKFK | LIYSASYR |
| | GKATLTVDT | GSGSGTDFTL | 64) | GKATLTVDTS | YSGVPDRF |
| | SSSTAYMQLS | TIRNVQSEDL | | SSTAYMQLSS | TGSGSGTD |
| | SLTSEDSAVY | AEYFCQQHN | | LTSEDSAVYY | FTLTIRNV |
| | YCARSPFDY | SYPFTFGGGT | | CARSPFDYW | QSEDLAEY |
| | WGQGTTLTV | KLEIK (SEQ | | GQGTTLTVSS | FCQQHNSY |
| | SS (SEQ ID | ID NO: 60) | | (SEQ ID NO: | PFTFGGGT |
| | NO: 53) | | | 53) | KLEIK (SEQ |
| | | | | | ID NO: 60) |
| Tandem 7 | EVQLLESGG | DIQMTQSPSS | GGGG | QVQLQQPGA | DVVMTQS |
| + 1-H01-1 | GLVQPGGSL | LSASVGDRV | SGGG | ELVKPGASV | QKFMSTSV |
| bispecific | RLSCAASGFP | TITCRASETIS | GSGG | KMSCKASGY | GDRVSVTC |
| | FGNYLMNW | NFLNWYQQ | GGS | TFTSYWMHW | KASQNVGT |
| | VRQAPGKGL | KPGKAPKLLI | (SEQ | VKQRPGQGL | NVAWYQQ |
| | EWVSVISGG | YTTSSLQRG | ID NO: | EWIGVIDPSN | KPGQSPKA |
| | GVSTYYADS | VPSRFSGSGS | 64) | SYTIYNQKFK | LIYSASYR |
| | VKGRFTISRD | GTDFTLTISS | | GKATLTVDTS | YSGVPDRF |
| | NSKNTLYLQ | LQPEDFATY | | SSTAYMQLSS | TGSGSGTD |
| | MNSLRAEDT | YCQQSYSVP | | LTSEDSAVYY | FTLTIRNV |
| | AVYYCARGP | WTFGGGTKV | | CARSPFDYW | QSEDLAEY |
| | SSWPAFDVW | EIK (SEQ ID | | GQGTTLTVSS | FCQQHNSY |
| | GQGTLVTVSS | NO: 6) | | (SEQ ID NO: | PFTFGGGT |
| | (SEQ ID NO: | | | 53) | KLEIK (SEQ |
| | 1) | | | | ID NO: 60) |
| Tandem | QVQLQQPGA | DVVMTQSQK | GGGG | EVQLLESGGG | DIQMTQSP |
| 1-H01-1 | ELVKPGASV | FMSTSVGDR | SGGG | LVQPGGSLRL | SSLSASVG |
| + 7 | KMSCKASGY | VSVTCKASQ | GSGG | SCAASGFPFG | DRVTITCR |
| bispecific | TFTSYWMHW | NVGTNVAW | GGS | NYLMNWVR | ASETISNFL |
| | VKQRPGQGL | YQQKPGQSP | (SEQ | QAPGKGLEW | NWYQQKP |
| | EWIGVIDPSN | KALIYSASYR | ID NO: | VSVISGGGVS | GKAPKLLI |
| | SYTIYNQKFK | YSGVPDRFT | 64) | TYYADSVKG | YTTSSLQR |
| | GKATLTVDT | GSGSGTDFTL | | RFTISRDNSK | GVPSRFSG |
| | SSSTAYMQLS | TIRNVQSEDL | | NTLYLQMNS | SGSGTDFT |
| | SLTSEDSAVY | AEYFCQQHN | | LRAEDTAVY | LTISSLQPE |
| | YCARSPFDY | SYPFTFGGGT | | YCARGPSSW | DFATYYCQ |
| | WGQGTTLTV | KLEIK (SEQ | | PAFDVWGQG | QSYSVPWT |
| | SS (SEQ ID | ID NO: 60) | | TLVTVSS | FGGGTKVE |
| | NO: 53) | | | (SEQ ID NO: 1) | IK (SEQ ID |
| | | | | | NO: 6) |

TABLE 3
Exemplary Tandem scFv-Fc Nucleic Acid Sequences

| | Variable | Variable | | Variable | Variable |
|---|---|---|---|---|---|
| | Heavy Chain | Light Chain | | Heavy Chain | Light Chain |
| | Nucleic Acid | Nucleic Acid | scFv1- | Nucleic Acid | Nucleic Acid |
| Antibody | Sequence 1 | Sequence 1 | scFv2 | Sequence 2 | Sequence 2 |
| ID | (VH1) | (VL1) | Linker | (VH2) | (VL2) |
| Tandem 7 | GAGGTGCAG | GACATCCAG | GGCG | GAGGTGCAG | GACATCCA |
| mono- | CTGCTGGAG | ATGACCCAG | GCGG | CTGCTGGAG | GATGACCC |
| specific | TCCGGCGGC | TCCCCTCCT | CGGC | TCCGGCGGC | AGTCCCCT |
| | GGCCTGGTG | CCCTGTCCG | TCCG | GGCCTGGTG | CCTCCCTG |
| | CAGCCCGGC | CCTCCGTGG | GCGG | CAGCCCGGC | TCCGCCTC |
| | GGCTCCCTG | GCGACAGGG | CGGC | GGCTCCCTG | CGTGGGC |
| | AGGCTGTCC | TGACCATCA | GGCT | AGGCTGTCC | GACAGGG |
| | TGCGCCGCC | CCTGCAGGG | CCGG | TGCGCCGCC | TGACCATC |
| | TCCGGCTTC | CCTCCGAGA | CGGC | TCCGGCTTCC | ACCTGCAG |
| | CCCTTCGGC | CCATCTCCA | GGCG | CCTTCGGCA | GGCCTCCG |
| | AACTACCTG | ACTTCCTGA | GCTC | ACTACCTGA | AGACCATC |
| | ATGAACTGG | ACTGGTACC | C | TGAACTGGG | TCCAACTT |
| | GTGAGGCAG | AGCAGAAGC | (SEQ | TGAGGCAGG | CCTGAACT |
| | GCCCCCGGC | CCGGCAAGG | ID NO: | CCCCCGGCA | GGTACCA |
| | AAGGGCCTG | CCCCCAAGC | 65) | AGGGCCTGG | GCAGAAG |
| | GAGTGGGTG | TGCTGATCT | | AGTGGGTGT | CCCGGCA |
| | TCCGTGATC | ACACCACCT | | CCGTGATCT | AGGCCCCC |
| | TCCGGCGGC | CCTCCCTGC | | CCGGCGGCG | AAGCTGCT |
| | GGCGTGTCC | AGAGGGGC | | GCGTGTCCA | GATCTACA |
| | ACCTACTAC | GTGCCCTCC | | CCTACTACG | CCACCTCC |
| | GCCGACTCC | AGGTTCTCC | | CCGACTCCG | TCCCTGCA |
| | GTGAAGGGC | GGCTCCGGC | | TGAAGGGCA | GAGGGGC |
| | AGGTTCACC | TCCGGCACC | | GGTTCACCA | GTGCCCTC |
| | ATCTCCAGG | GACTTCACC | | TCTCCAGGG | CAGGTTCT |
| | GACAACTCC | CTGACCATC | | ACAACTCCA | CCGGCTCC |
| | AAGAACACC | TCCTCCCTG | | AGAACACCC | GGCTCCGG |
| | CTGTACCTG | CAGCCCGAG | | TGTACCTGC | CACCGACT |
| | CAGATGAAC | GACTTCGCC | | AGATGAACT | TCACCCTG |
| | TCCCTGAGG | ACCTACTAC | | CCCTGAGGG | ACCATCTC |
| | GCCGAGGAC | TGCCAGCAG | | CCGAGGACA | CTCCCTGC |
| | ACCGCCGTG | TCCTACTCC | | CCGCCGTGT | AGCCCGA |
| | TACTACTGC | GTGCCCTGG | | ACTACTGCG | GGACTTCG |
| | GCCAGGGGC | ACCTTCGGC | | CCAGGGGCC | CCACCTAC |
| | CCCTCCTCCT | GGCGGCACC | | CCTCCTCCTG | TACTGCCA |
| | GGCCCGCCT | AAGGTGGAG | | GCCCGCCTT | GCAGTCCT |
| | TCGACGTGT | ATCAAG | | CGACGTGTG | ACTCCGTG |
| | GGGGCCAGG | (SEQ ID NO: | | GGGCCAGGG | CCCTGGAC |
| | GCACCCCTG | )93 | | CACCCCTGG | CTTCGGCG |
| | GTGACCGTG | | | TGACCGTGT | GCGGCAC |
| | TCCTCC | | | CCTCC (SEQ | CAAGGTG |
| | (SEQ ID NO: | | | ID NO: 94) | GAGATCA |
| | 92) | | | | AG (SEQ |
| | | | | | ID NO: 95) |
| Tandem | CAGGTGCAG | GACGTGGTG | GGCG | CAGGTGCAG | GACGTGGT |
| 1-H01-1 | CTGCAGCAG | ATGACCCAG | GCGG | CTGCAGCAG | GATGACCC |
| mono- | CCCGGCGCC | TCCCAGAAG | CGGC | CCCGGCGCC | AGTCCCAG |
| specific | GAGCTGGTG | TTCATGTCC | TCCG | GAGCTGGTG | AAGTTCAT |
| | AAGCCCGGC | ACCTCCGTG | GCGG | AAGCCCGGC | GTCCACCT |
| | GCCTCCGTG | GGCGACAGG | CGGC | GCCTCCGTG | CCGTGGGC |
| | AAGATGTCC | GTGTCCGTG | GGCT | AAGATGTCC | GACAGGG |
| | TGCAAGGCC | ACCTGCAAG | CCGG | TGCAAGGCC | TGTCCGTG |
| | TCCGGCTAC | GCCTCCCAG | CGGC | TCCGGCTAC | ACCTGCAA |
| | ACCTTCACC | AACGTGGGC | GGCG | ACCTTCACCT | GGCCTCCC |
| | TCCTACTGG | ACCAACGTG | GCTC | CCTACTGGA | AGAACGT |
| | ATGCACTGG | GCCTGGTAC | C | TGCACTGGG | GGGCACC |
| | GTGAAGCAG | CAGCAGAAG | (SEQ | TGAAGCAGA | AACGTGG |
| | AGGCCCAGC | CCCGGCCAG | ID NO:) | GGCCCAGCC | CCTGGTAC |
| | CAGGGCCTG | TCCCCCAAG | | AGGGCCTGG | CAGCAGA |
| | GAGTGGATC | GCCCTGATC | | AGTGGATCG | AGCCCGG |
| | GGCGTGATC | TACTCCGCC | | GCGTGATCG | CCAGTCCC |
| | GACCCCTCC | TCCTACAGG | | ACCCCTCCA | CCAAGGC |
| | AACTCCTAC | TACTCCGGC | | ACTCCTACA | CCTGATCT |
| | ACCATCTAC | GTGCCCGAC | | CCATCTACA | ACTCCGCC |
| | AACCAGAAG | AGGTTCACC | | ACCAGAAGT | TCCTACAG |
| | TTCAAGGGC | GGCTCCGGC | | TCAAGGGCA | GTACTCCG |
| | AAGGCCACC | TCCGGCACC | | AGGCCACCC | GCGTGCCC |
| | CTGACCCTG | GACTTCACC | | TGACCCTGG | GACAGGTT |
| | GACACCTCC | CTGACCATC | | ACACCTCCT | CACCGGCT |
| | TCCTCCACC | AGGAACGTG | | CCTCCACCG | CCGGCTCC |
| | GCCTACATG | CAGTCCGAG | | CCTACATGC | GGCACCG |
| | CAGCTGTGC | GACCTGGCC | | AGCTGTGCT | ACTTCACC |
| | TCCCTGACC | GAGTACTTC | | CCCTGACCT | CTGACCAT |
| | TCCGAGGAC | TGCCAGCAG | | CCGAGGACT | CAGGAAC |
| | TCCGCCGTG | CACAACCTC | | CCGCCGTGT | GTGCAGTC |
| | TACTACTGC | CTACCCCTT | | ACTACTGCC | CGAGGAC |
| | CAGGTCCCC | CACCTTCGG | | AGGTCCCCC | CTGGCCGA |
| | CTTCGACTA | CGGCGGCAC | | TTCGACTACT | GTACTTCT |
| | CTGGGGCCA | CAAGCTGGA | | GGGGCCAGG | GCCAGCA |
| | GGGCACCAA | GATCAAG | | GCACCAACC | GCACAAC |
| | CCTGACCGT | (SEQ ID NO: | | TGACCGTGT | CTCCTACC |
| | GTGCTCC | 97) | | GCTCC (SEQ | CCTTCACC |
| | (SEQ ID NO: | | | ID NO: 98) | TTCGGCGG |
| | 96) | | | | CGGCACC |
| | | | | | AAGCTGG |
| | | | | | AGATCAA |
| | | | | | G (SEQ |
| | | | | | ID NO: 99) |
| Tandem 7 | GAGGTGCAG | GACATCCAG | GGCG | CAGGTGCAG | GACGTGGT |
| + 1-H01- | CTGCTGGAG | ATGACCCAG | GCGG | CTGCAGCAG | GATGACCC |
| 1 | TCCGGCGGC | TCCCCTCCT | CGGC | CCCGGCGCC | AGTCCCAG |
| bispecific | GGCCTGGTG | CCCTGTCCG | TCCG | GAGCTGGTG | AAGTTCAT |
| | CAGCCCGGC | CCTCCGTGG | GCGG | AAGCCCGGC | GTCCACCT |
| | GGCTCCCTG | GCGACAGGG | CGGC | GCCTCCGTG | CCGTGGGC |
| | AGGCTGTCC | TGACCATCA | GGCT | AAGATGTCC | GACAGGG |
| | TGCGCCGCC | CCTGCAGGG | CCGG | TGCAAGGCC | TGTCCGTG |
| | TCCGGCTTC | CCTCCGAGA | CGGC | TCCGGCTAC | ACCTGCAA |
| | CCCTTCGGC | CCATCTCCA | GGCG | ACCTTCACCT | GGCCTCCC |
| | AACTACCTG | ACTTCCTGA | GCTC | CCTACTGGA | AGAACGT |
| | ATGAACTGG | ACTGGTACC | C | TGCACTGGG | GGGCACC |
| | GTGAGGCAG | AGCAGAAGC | (SEQ | TGAAGCAGA | AACGTGG |
| | GCCCCCGGC | CCGGCAAGG | ID NO:) | GGCCCAGCC | CCTGGTAC |
| | AAGGGCCTG | CCCCCAAGC | | AGGGCCTGG | CAGCAGA |
| | GAGTGGGTG | TGCTGATCT | | AGTGGATCG | AGCCCGG |
| | TCCGTGATC | ACACCACCT | | GCGTGATCG | CCAGTCCC |
| | TCCGGCGGC | CCTCCCTGC | | ACCCCTCCA | CCAAGGC |
| | GGCGTGTCC | AGAGGGGC | | ACTCCTACA | CCTGATCT |
| | ACCTACTAC | GTGCCCTCC | | CCATCTACA | ACTCCGCC |
| | GCCGACTCC | AGGTTCTCC | | ACCAGAAGT | TCCTACAG |
| | GTGAAGGGC | GGCTCCGGC | | TCAAGGGCA | GTACTCCG |
| | AGGTTCACC | TCCGGCACC | | AGGCCACCC | GCGTGCCC |
| | ATCTCCAGG | GACTTCACC | | TGACCCTGG | GACAGGTT |
| | GACAACTCC | CTGACCATC | | ACACCTCCT | CACCGGCT |
| | AAGAACACC | TCCTCCCTG | | CCTCCACCG | CCGGCTCC |
| | CTGTACCTG | CAGCCCGAG | | CCTACATGC | GGCACCG |
| | CAGATGAAC | GACTTCGCC | | AGCTGTGCT | ACTTCACC |
| | TCCCTGAGG | ACCTACTAC | | CCCTGACCT | CTGACCAT |
| | GCCGAGGAC | TGCCAGCAG | | CCGAGGACT | CAGGAAC |
| | ACCGCCGTG | TCCTACTCC | | CCGCCGTGT | GTGCAGTC |
| | TACTACTGC | GTGCCCTGG | | ACTACTGCC | CGAGGAC |
| | GCCAGGGGC | ACCTTCGGC | | AGGTCCCCC | CTGGCCGA |
| | CCCTCCTCCT | GGCGGCACC | | TTCGACTACT | GTACTTCT |
| | GGCCCGCCT | AAGGTGGAG | | GGGGCCAGG | GCCAGCA |
| | TCGACGTGT | ATCAAG | | GCACCAACC | GCACAAC |
| | GGGGCCAGG | (SEQ ID NO: | | TGACCGTGT | CTCCTACC |
| | GCACCCCTG | 101) | | GCTCC (SEQ | CCTTCACC |
| | GTGACCGTG | | | ID NO: 102) | TTCGGCGG |
| | TCCTCC | | | | CGGCACC |
| | (SEQ ID NO: | | | | AAGCTGG |
| | 100) | | | | AGATCAA |
| | | | | | G (SEQ |
| | | | | | ID NO: 103) |
| Tandem | CAGGTGCAG | GACGTGGTG | GGCG | GAGGTGCAG | GACATCCA |
| 1-H01-1 | CTGCAGCAG | ATGACCCAG | GCGG | CTGCTGGAG | GATGACCC |
| + 7 | CCCGGCGCC | TCCCAGAAG | CGGC | TCCGGCGGC | AGTCCCCT |
| bispecific | GAGCTGGTG | TTCATGTCC | TCCG | GGCCTGGTG | CCTCCCTG |
| | AAGCCCGGC | ACCTCCGTG | GCGG | CAGCCCGGC | TCCGCCTC |
| | GCCTCCGTG | GGCGACAGG | CGGC | GGCTCCCTG | CGTGGGC |
| | AAGATGTCC | GTGTCCGTG | GGCT | AGGCTGTCC | GACAGGG |
| | TGCAAGGCC | ACCTGCAAG | CCGG | TGCGCCGCC | TGACCATC |
| | TCCGGCTAC | GCCTCCCAG | CGGC | TCCGGCTTCC | ACCTGCAG |
| | ACCTTCACC | AACGTGGGC | GGCG | CCTTCGGCA | GGCCTCCG |
| | TCCTACTGG | ACCAACGTG | GCTC | ACTACCTGA | AGACCATC |
| | ATGCACTGG | GCCTGGTAC | C | TGAACTGGG | TCCAACTT |
| | GTGAAGCAG | CAGCAGAAG | (SEQ | TGAGGCAGG | CCTGAACT |
| | AGGCCCAGC | CCCGGCCAG | ID NO:) | CCCCCGGCA | GGTACCA |
| | CAGGGCCTG | TCCCCCAAG | | AGGGCCTGG | GCAGAAG |
| | GAGTGGATC | GCCCTGATC | | AGTGGGTGT | CCCGGCA |
| | GGCGTGATC | TACTCCGCC | | CCGTGATCT | AGGCCCCC |
| | GACCCCTCC | TCCTACAGG | | CCGGCGGCG | AAGCTGCT |
| | AACTCCTAC | TACTCCGGC | | GCGTGTCCA | GATCTACA |
| | ACCATCTAC | GTGCCCGAC | | CCTACTACG | CCACCTCC |
| | AACCAGAAG | AGGTTCACC | | CCGACTCCG | TCCCTGCA |
| | TTCAAGGGC | GGCTCCGGC | | TGAAGGGCA | GAGGGGC |
| | AAGGCCACC | TCCGGCACC | | GGTTCACCA | GTGCCCTC |
| | CTGACCCTG | GACTTCACC | | TCTCCAGGG | CAGGTTCT |
| | GACACCTCC | CTGACCATC | | ACAACTCCA | CCGGCTCC |
| | TCCTCCACC | AGGAACGTG | | AGAACACCC | GGCTCCGG |
| | GCCTACATG | CAGTCCGAG | | TGTACCTGC | CACCGACT |
| | CAGCTGTGC | GACCTGGCC | | AGATGAACT | TCACCCTG |
| | TCCCTGACC | GAGTACTTC | | CCCTGAGGG | ACCATCTC |
| | TCCGAGGAC | TGCCAGCAG | | CCGAGGACA | CTCCCTGC |
| | TCCGCCGTG | CACAACCTC | | CCGCCGTGT | AGCCCGA |
| | TACTACTGC | CTACCCCTT | | ACTACTGCG | GGACTTCG |
| | CAGGTCCCC | CACCTTCGG | | CCAGGGGCC | CCACCTAC |
| | CTTCGACTA | CGGCGGCAC | | CCTCCTCCTG | TACTGCCA |
| | CTGGGGCCA | CAAGCTGGA | | GCCCGCCTT | GCAGTCCT |
| | GGGCACCAA | GATCAAG | | CGACGTGTG | ACTCCGTG |
| | CCTGACCGT | (SEQ ID NO: | | GGGCCAGGG | CCCTGGAC |
| | GTGCTCC | 105) | | CACCCCTGG | CTTCGGCG |
| | (SEQ ID NO: | | | TGACCGTGT | GCGGCAC |
| | 104) | | | CCTCC (SEQ | CAAGGTG |
| | | | | ID NO: 106) | GAGATCA |
| | | | | | AG (SEQ |
| | | | | | ID NO: 107) |

In exemplary embodiments, provided herein is a tandem scFv-Fc PD-1 agonist antibody with a scFv1 and a scFv2 on each antibody arm, wherein the first heavy chain variable domain (VH1) of the antibody comprises the amino acid sequence of SEQ ID NO: 1; wherein the second heavy chain variable domain (VH2) of the antibody comprises the amino acid sequence of SEQ ID NO: 1; wherein the first light chain variable domain (VL1) of the antibody comprises the amino acid sequence of SEQ ID NO: 6; and wherein the second light chain variable domain (VL2) of the antibody comprises the amino acid sequence of SEQ ID NO: 6; wherein the scFv1 and scFv2 are linked with a linker comprising the following amino acid sequence: GGGGSGGGGSGGGGS (SEQ ID NO: 64).

In exemplary embodiments, provided herein is a tandem scFv-Fc PD-1 agonist antibody with a scFv1 and a scFv2 on each antibody arm, wherein the first heavy chain variable domain (VH1) of the antibody comprises the amino acid sequence of SEQ ID NO: 53; wherein the second heavy chain variable domain (VH2) of the antibody comprises the amino acid sequence of SEQ ID NO: 53; wherein the first light chain variable domain (VL1) of the antibody comprises the amino acid sequence of SEQ ID NO: 60; and wherein the second light chain variable domain (VL2) of the antibody comprises the amino acid sequence of SEQ ID NO: 60; wherein the scFv1 and scFv2 are linked with a linker comprising the following amino acid sequence: GGGGSGGGGSGGGGS (SEQ ID NO: 64).

In exemplary embodiments, provided herein is a tandem scFv-Fc PD-1 agonist antibody with a scFv1 and a scFv2 on each antibody arm, wherein the first heavy chain variable domain (VH1) of the antibody comprises the amino acid sequence of SEQ ID NO: 1; wherein the second heavy chain variable domain (VH2) of the antibody comprises the amino acid sequence of SEQ ID NO: 53; wherein the first light chain variable domain (VL1) of the antibody comprises the amino acid sequence of SEQ ID NO: 6; and wherein the second light chain variable domain (VL2) of the antibody comprises the amino acid sequence of SEQ ID NO: 60; wherein the scFv1 and scFv2 are linked with a linker comprising the following amino acid sequence: GGGGSGGGGSGGGGS (SEQ ID NO: 64).

In exemplary embodiments, provided herein is a tandem scFv-Fc PD-1 agonist antibody with a scFv1 and a scFv2 on each antibody arm, wherein the first heavy chain variable domain (VH1) of the antibody comprises the amino acid sequence of SEQ ID NO: 53; wherein the second heavy chain variable domain (VH2) of the antibody comprises the amino acid sequence of SEQ ID NO: 1; wherein the first light chain variable domain (VL1) of the antibody comprises the amino acid sequence of SEQ ID NO: 60; and wherein the second light chain variable domain (VL2) of the antibody comprises the amino acid sequence of SEQ ID NO: 6; wherein the scFv1 and scFv2 are linked with a linker comprising the following amino acid sequence: GGGGSGGGGSGGGGS (SEQ ID NO: 64).

As depicted in FIG. 11, the Fc domain of the tandem scFv-Fc PD-1 agonist antibodies of disclosure may be human IgG1, human IgG2, human IgG3, or. human IgG4.

II. Uses of PD-1 Agonist Antibodies.

A. Therapeutic PD-1 Agonist Antibodies.

In some embodiments, the PD-1 agonist antibodies provided herein are useful for the treatment of a disease or condition involving an immune response.

In some embodiments, the PD-1 agonist antibodies provided herein are useful for the treatment of an autoimmune disease. An autoimmune disease consists of a potentially harmful immune response to a self-antigen. Examples of autoimmune diseases include: alopecia, ankylosing spondylitis, atopic dermatitis, celiac disease, Crohn's disease, cutaneous lupus erythematosus (CLE), lupus nephritis, multiple sclerosis, neuromyelitis optica, psoriasis, psoriatic arthritis, rheumatoid arthritis, scleroderma, Sjogren's syndrome, systemic lupus, systemic lupus erythematosus (SLE), temporal arteritis, type I diabetes, ulcerative colitis, uveitis, and vitiligo.

In some embodiments, the PD-1 agonist antibodies provided herein are useful for the treatment of a hyperinflammatory disease. A hyperinflammatory disease consists of a potentially harmful overstimulated immune response. Examples of hyperinflammatory diseases include: chronic allergy, hypersensitivity vasculitis, and T cell hypersensitivity disease.

B. Administration of Therapeutic PD-1 Agonist Antibodies.

The in vivo administration of the therapeutic PD-1 agonist antibodies described herein may be carried out intravenously, intramuscularly, subcutaneously, topically, orally, transdermally, intraperitoneally, intraorbitally, intrathecally, intraventricularly, intranasally, transmucosally, through implantation, or through inhalation. Intravenous administration may be carried out via injection or infusion. In some embodiments, the PD-1 agonist antibodies of the disclosure are administered intravenously. In some embodiments, the PD-1 agonist antibodies of the disclosure are administered subcutaneously. Administration of the therapeutic PD-1 agonist antibodies may be performed with any suitable excipients, carriers, or other agents to provide suitable or improved tolerance, transfer, delivery, and the like.

### EXAMPLES

### Example 1: PD-1 Agonist Discovery Based on Engineered MEM-Nanoparticle Immunizations

MEMs were designed based on the epitope identified by SEQ ID NO: 62 and then conjugated to nanoparticles to steer B-cell antibody production towards said epitope and away from the PD-L1 binding site, FIG. 1A. MEMs conjugated to ferritin nanoparticles were identified by Coomassie-based western blots and were found to contain approximately 20-30 MEMs per nanoparticle, FIG. 1B. The MEM-nanoparticles demonstrated nanomolar binding affinity with PD1AB6 using Surface Plasmon Resonance (SPR). BALB/c mice were then immunized over a 5-week period with alternating doses of the engineered MEM-nanoparticle and/or full-length PD-1 suspended in adjuvant, with a final boost containing a combination of the two, FIG. 1C. Mouse serum was collected, yielding strong PD-1 binding measured via ELISA, FIG. 1D.

### Example 2: Top Monoclonal Hybridoma Produced Antibody Demonstrates Strong PD-1 Binding and Agonism

Hybridomas were created from the immunized mouse B-cells using standard electro cell fusion methods. The resulting 27A5 antibody and several others were generated from monoclonal hybridomas and demonstrated strong PD-1 binding via ELISA, FIG. 2A. 27A5 was then further assessed in vitro for PD-1 binding and competition binding for PD-1 using SPR, FIG. 2B. 27A5 demonstrated a KD of 55 nM and blocked the PD1AB6 binding site but not the Pembrolizumab binding site. PD-1 avidity was also measured for 27A5 via SPR which demonstrated strong binding with a KD of 5 pM, FIG. 2C. 27A5 was then assessed for in vitro functionality at PD-1. PathHunter® Checkpoint Signaling Assays were performed to measure PD-1 agonism and antagonism, producing the resulting concentration/response curves FIG. 2D. 27A5 demonstrated comparable agonism to the control antibody and no antagonism.

### Example 3: Antibodies Generated from In Vitro scFv Library Demonstrate Strong PD-1 Binding

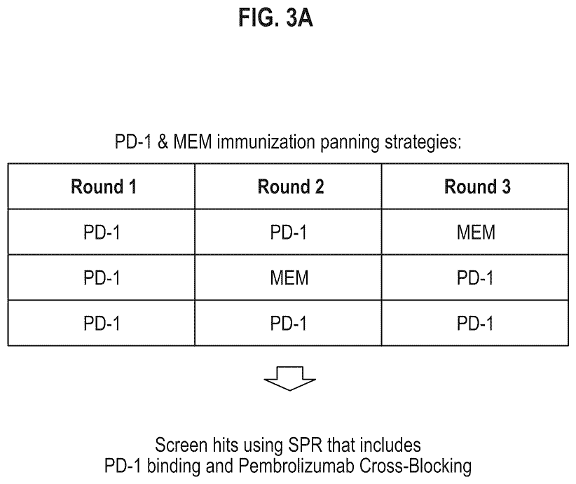

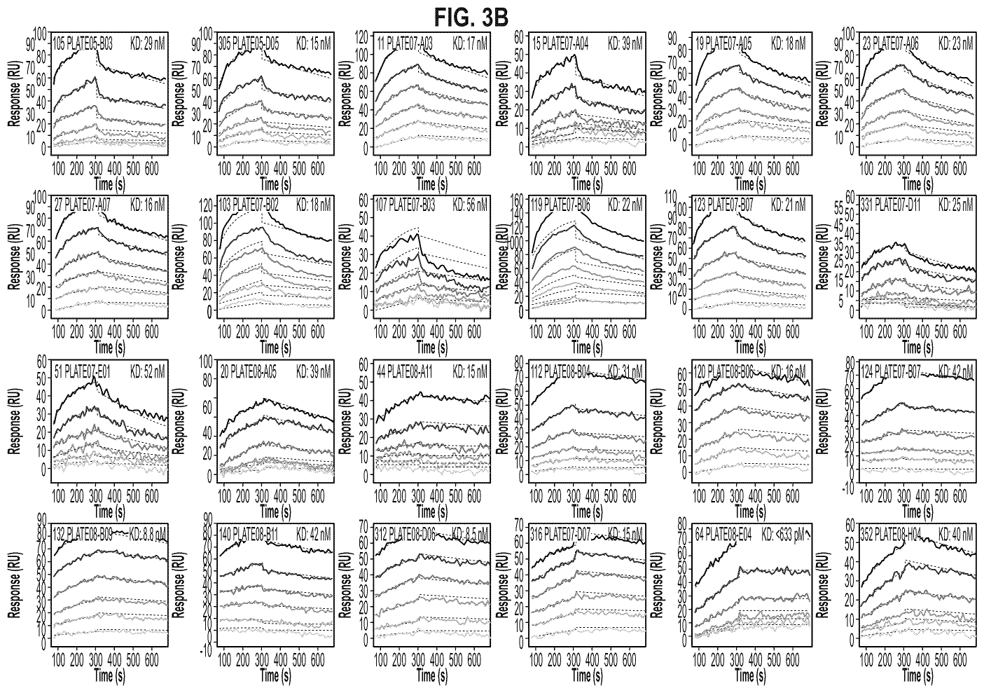

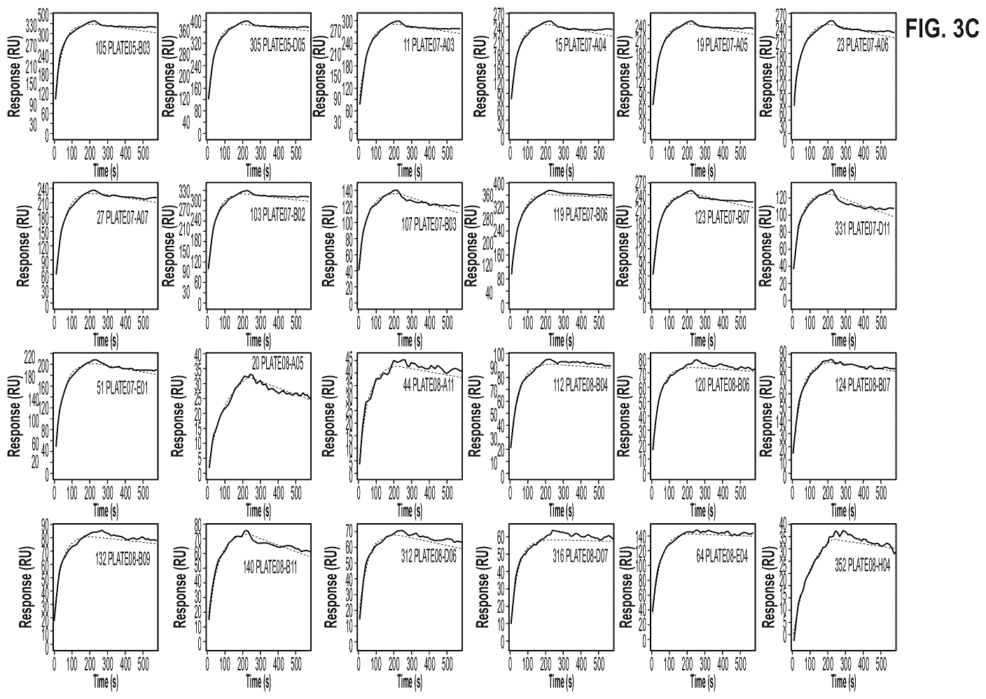

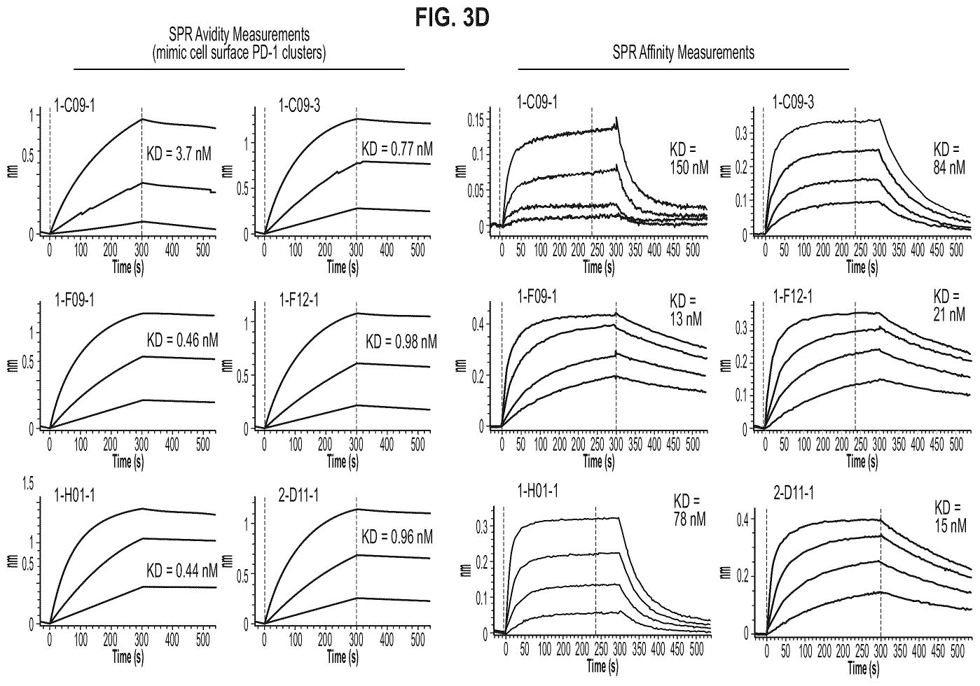

Mouse serum collected from the previous immunizations was additionally used to construct an in vitro scFv library using standard molecular cloning techniques and phage display. The library was subjected to three rounds of phage panning against full length PD-1 and the MEM-nanoparticle in the orders listed in FIG. 3A. The resulting isolated clones were then screened for PD-1 binding affinity and Pembrolizumab competition binding using SPR, FIGS. 3B-3C. Exemplary antibodies were identified from the scFv in vitro library which bound to PD-1 at a high affinity and avidity using SPR, FIG. 3D.

### Example 4: Selected Antibodies Generated from In Vitro scFv Library Demonstrate Agonism at the Intended Epitope

The selected antibodies were then evaluated for PD-1 agonism and antagonism using the PathHunter® Checkpoint Signaling Assay, FIGS. 4A-4B. The resulting agonist concentration-response curves were utilized to obtain EC50 values for each antibody, yielding agonist activity that was comparable or higher to the control antibody. The resulting antagonist concentration response curves were utilized to obtain IC50 values, which were not detectable for all six antibodies, demonstrating a lack of PD-1 antagonism. The six antibodies were then further evaluated for competitive binding of both the PD1AB6 binding site and the Pembrolizumab binding site, FIG. 4C. All six antibodies demonstrated a complete blockade of PD1AB6 binding and no blockage of Pembrolizumab binding. FIG. 4D lists the compiled agonism, avidity, and affinity values for all six antibodies.

### Example 5: Selected Antibodies Generated by PD-1/MEM-Nanoparticle Panning Strategy of Naïve Library Demonstrate Strong PD-1 Binding

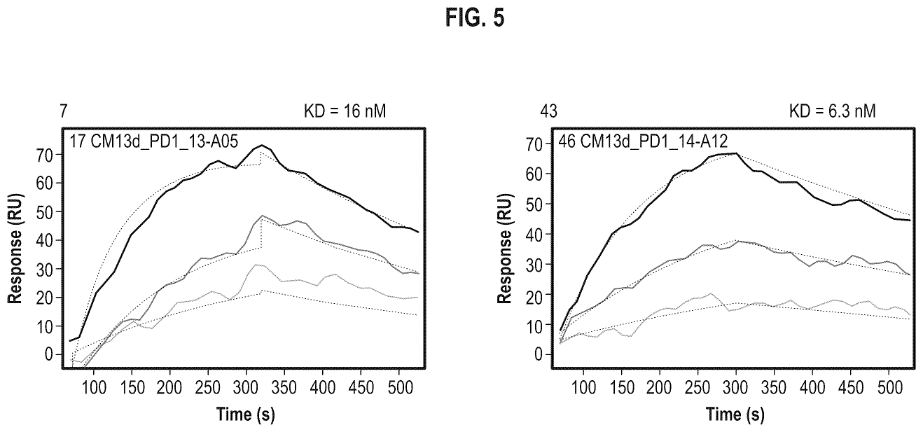

MEM-nanoparticles and full-length PD-1 were then used in a phage panning strategy of a naïve antibody library. Selected antibodies 7 and 43 were generated and further evaluated in vitro for PD-1 binding affinity. FIG. 5 shows SPR response curves for both antibodies at three concentrations. KD values were calculated from the SPR response curves and found to be in the nanomolar range for both antibodies.

### Example 6: PD-1 Agonist Activity of Top Antibodies Generated by Naïve Library Panning Strategy

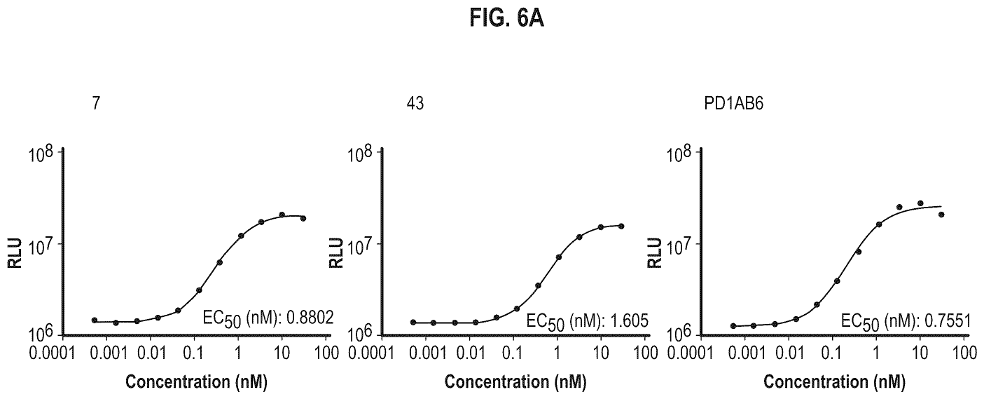

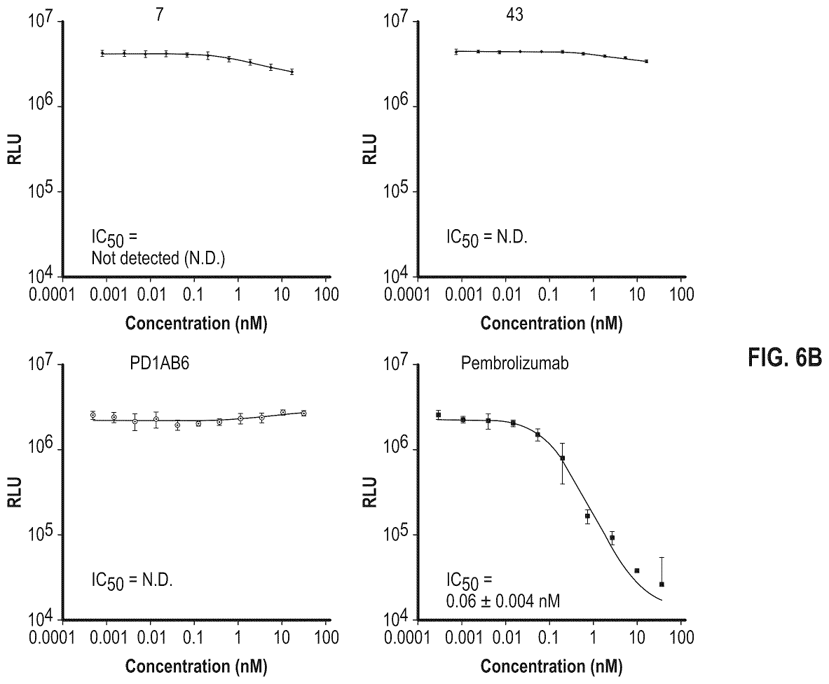

Antibodies 7 and 43 were then evaluated for PD-1 agonism in vitro. FIG. 6A shows the results of the agonist assay as log scale concentration-response curves. EC50 values were calculated, demonstrating a higher relative potency when compared to the control antibody. Both antibodies were additionally evaluated for PD-1 antagonism in vitro. FIG. 6B shows the results of the antagonist assay as log scale concentration-response curves. IC 50 values were not detectable for either antibody which showed an equivalent lack of antagonism when compared to the negative control antibody. Pembrolizumab was used as a positive control demonstrating an IC50 of 0.06 nM.

### Example 7: PD-1 Binding Affinity of Selected Antibodies Generated by AI/Mammalian Display Strategy

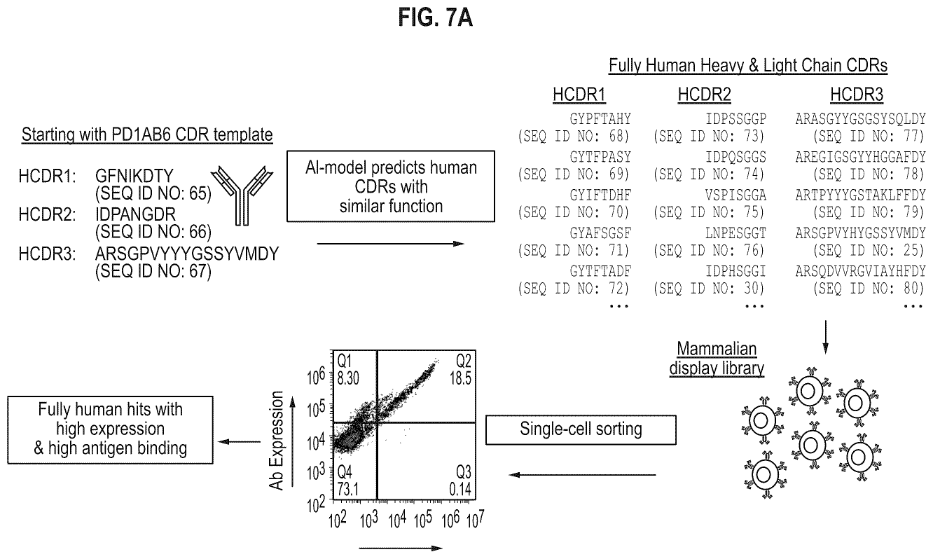

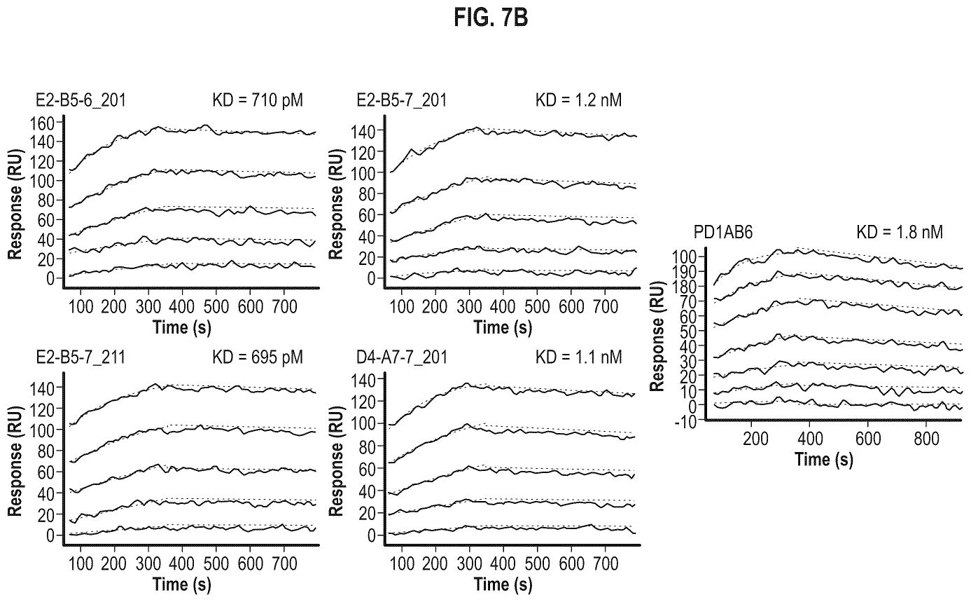

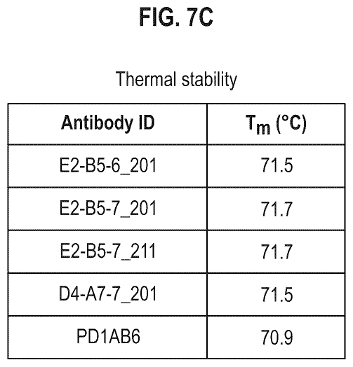

Fully humanized PD-1 agonist antibody CDRs were generated from AI-model predictions beginning from reference CDR antibody templates of PD1AB6, FIG. 7A. Mammalian display libraries were then created, and single cell sorted, using FACS, to select for antibodies with favorable binding properties and developability. Selected antibodies were evaluated in vitro for PD-1 binding affinity. FIG. 7B shows SPR response binding curves for the selected antibodies at five concentrations. KD values were calculated from the SPR response curves and found to be within pico and nanomolar ranges, demonstrating higher affinities when compared to PD1AB6. FIG. 7C shows calculated thermal stability for the selected antibodies demonstrating comparable Tm (° C.) values when compared to PD1AB6.

### Example 8: AI/Mammalian Display Generated Antibodies Demonstrate PD-1 Agonism at Intended Epitope

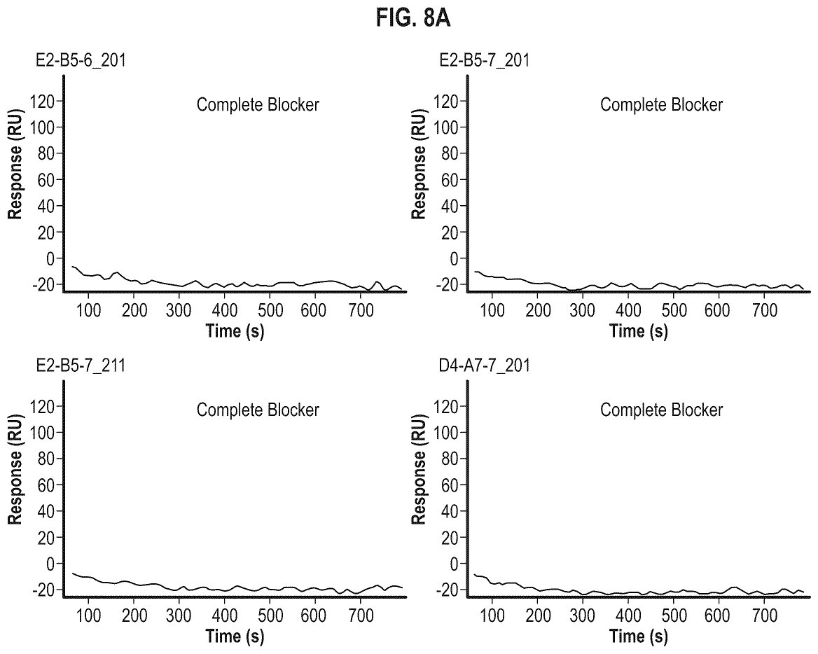

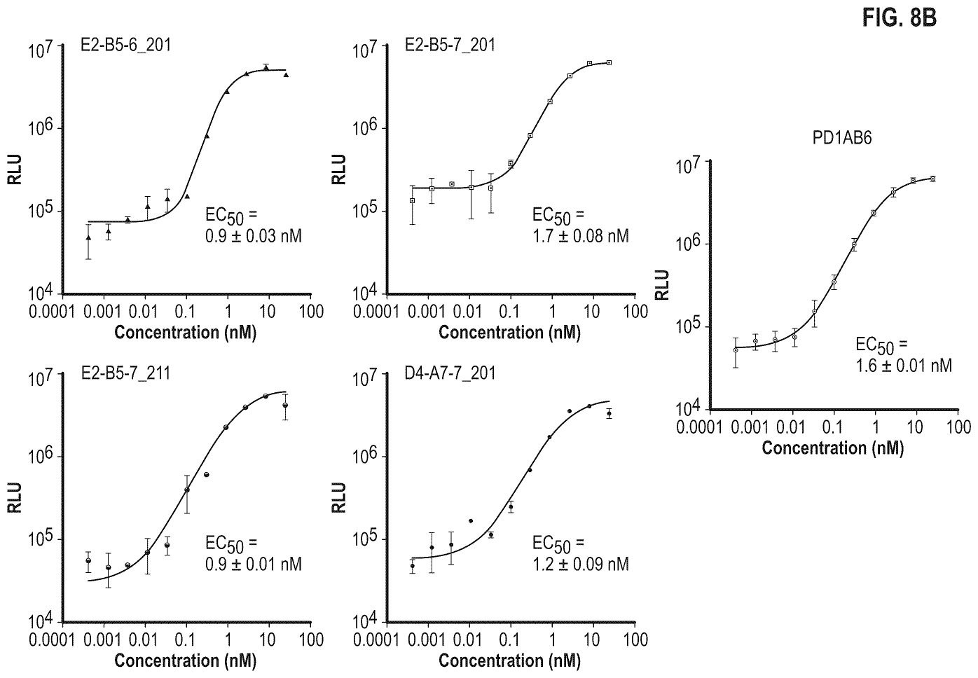

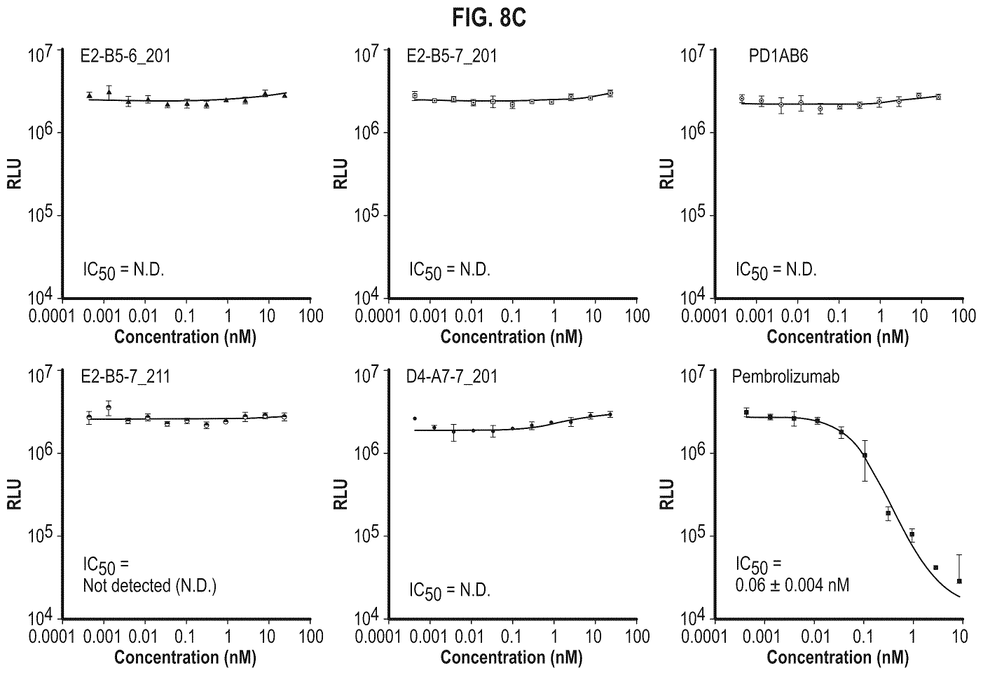

The selected antibodies were then tested in vitro for their ability to compete with PD1AB6 binding of PD-1. FIG. 8A shows SPR response curves, demonstrating a lack of binding of the PD1AB6, showing that all four antibodies demonstrate specific binding to the intended PD-1 epitope. The selected antibodies were then evaluated for PD-1 agonism in vitro. FIG. 8B shows the results of the agonist assay as log scale concentration-response curves. EC50 values were calculated, demonstrating equivalent or higher relative potency when compared to PD1AB6. The selected antibodies were then evaluated for PD-1 antagonism in vitro. FIG. 8C shows the results of the antagonist assay as log scale concentration-response curves. IC50 values were not detectable for all four antibodies which showed an equivalent lack of antagonism when compared to PD1AB6. Pembrolizumab was used as a positive control demonstrating an IC50 value of 0.06 nM.

### Example 9: Human Primary Cell Assays for Top In-Vitro Reporter Assay Clones

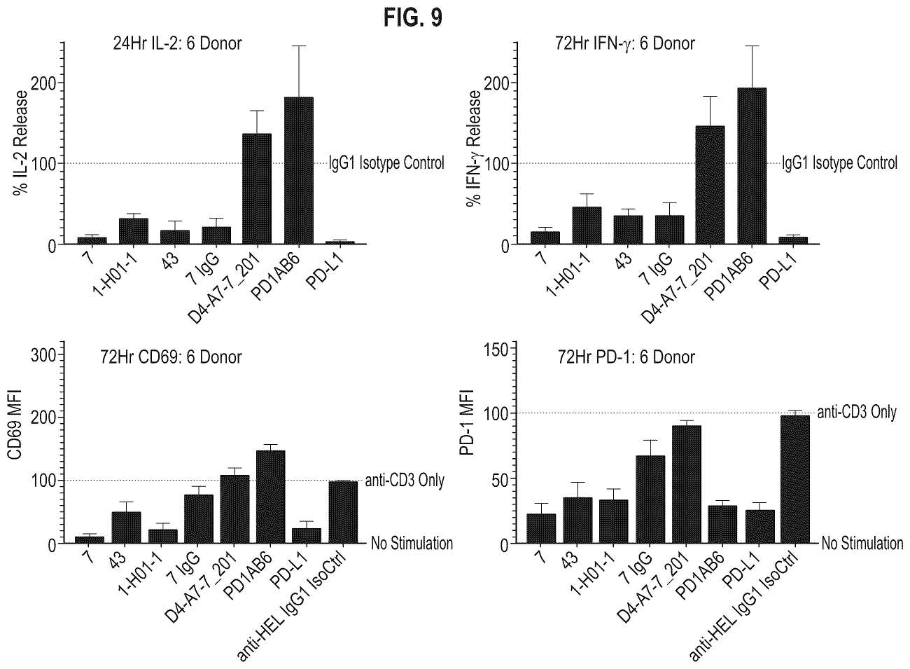

Antibody clones 7, 43, 1-H01-1 and D4-A7-7_201 were selected for human primary CD4 T-cell cytokine release and activation marker assays, these four antibodies were selected based on their PD-1 agonist in-vitro reporter assay EC50 values. Human PBMCs were isolated from 7-donors and activated with 5 ug/mL PHA for 48Hr to upregulate PD-1 expression. CD4 T-cells were then purified from the pre-stimulated PBMCs and plated at a uniform density on 96-well plates that were serially coated with 3 ug/mL OKT3 and a titration series of each test article. Supernatants were collected to measure IL-2 at 24Hr and IFN-gamma at 72Hr. CD4 T-cells were collected at 72Hr and analyzed by flow cytometry for PD-1 and CD69 activation biomarkers. FIG. 9 shows the results of the 7-donor CD4 T-cell cytokine release and activation marker assay. Clones 7, 43, 1-H01-1 and D4-A7-7_201 attenuate IL-2, IFN-gamma cytokines and CD69 T-cell activation marker significantly more than PD1AB6. Clones 7, 43 and 1-H01-1 show significant downregulation of PD-1 expression relative to isotype control and comparable to PD1AB6, suggesting that these clones potently agonize the PD-1 pathway, thereby triggering downregulation of PD-1.

### Example 10: Epitope Binning

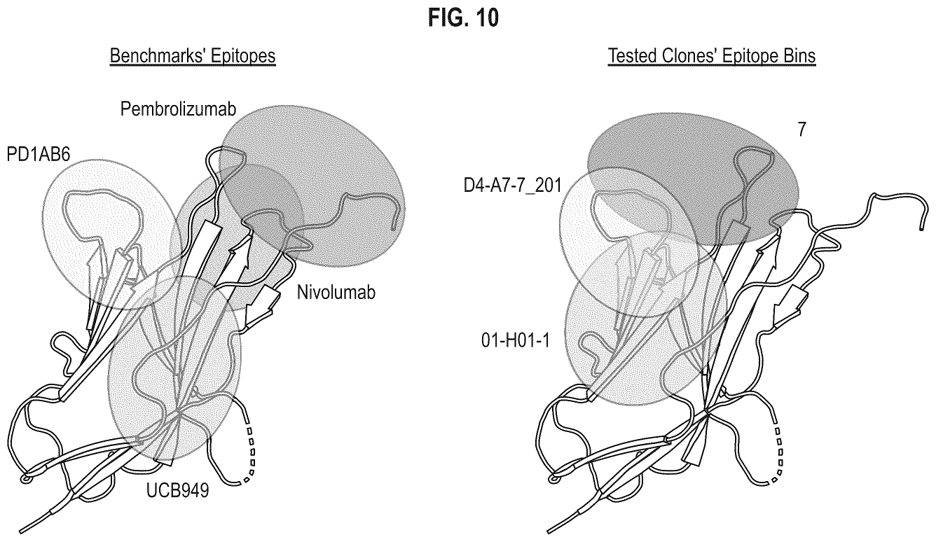

Antibody clones 7, 1-H01-1 and D4-A7-7_201 were selected for epitope binning based on their in vitro reporter and human primary cell assay results. Epitope binning was performed by testing the ability of clones 7, 1-H01-1 and D4-A7-7_201 to compete in SPR binding with each other and benchmark antibodies PD1AB6, Nivolumab, Pembrolizumab, and UCB949 that have known PD-1 binding epitopes. FIG. 10 shows the epitope binning results which indicate clones 7, 1-H01-1 and D4-A7-7_201 bind distinct regions on PD-1.

### Example 11: Tandem scFv-Fc Antibody Format

Antibody clones 7 and 1-H01-1 were selected for testing in what is designated herein as a tandem scFv-Fc format, with two scFv binding sites in tandem on each antibody arm linked by a linker (in this example, a flexible linker), and a total of four scFv binding sites in a single scFv-Fc formatted antibody, see exemplary structure in FIG. 11.

Without being held to theory or mechanism, PD-1 agonism is believed to be driven by clustering of multiple PD-1 receptors, and thus a tandem scFv-Fc antibody with greater than two PD-1 binding sites may exhibit stronger PD-1 agonism when compared to the analogous two binding site antibody.

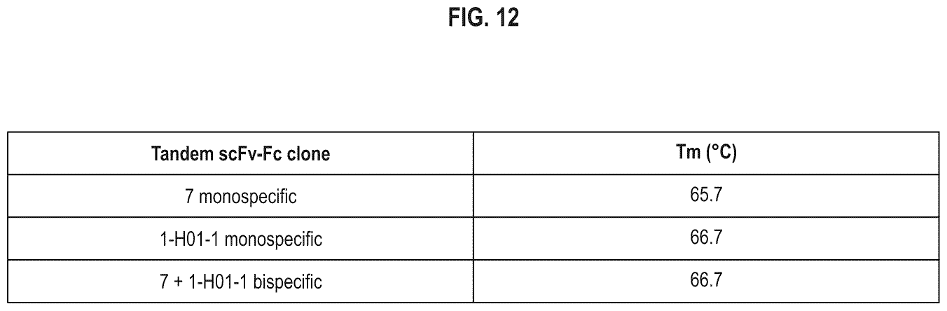

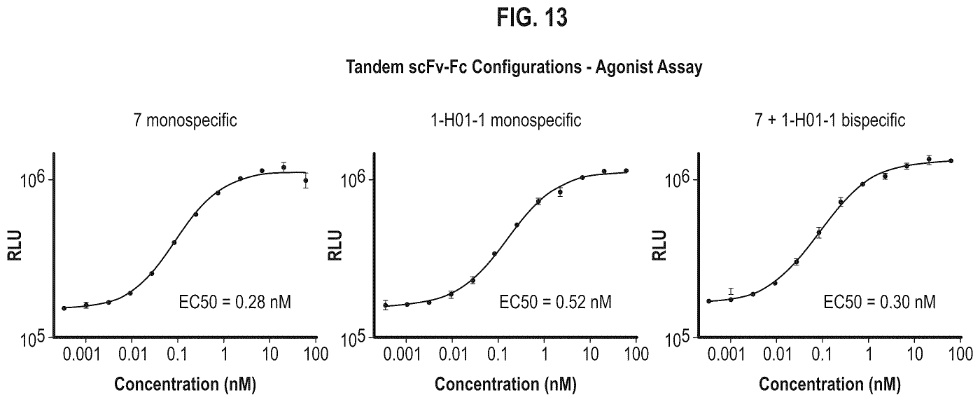

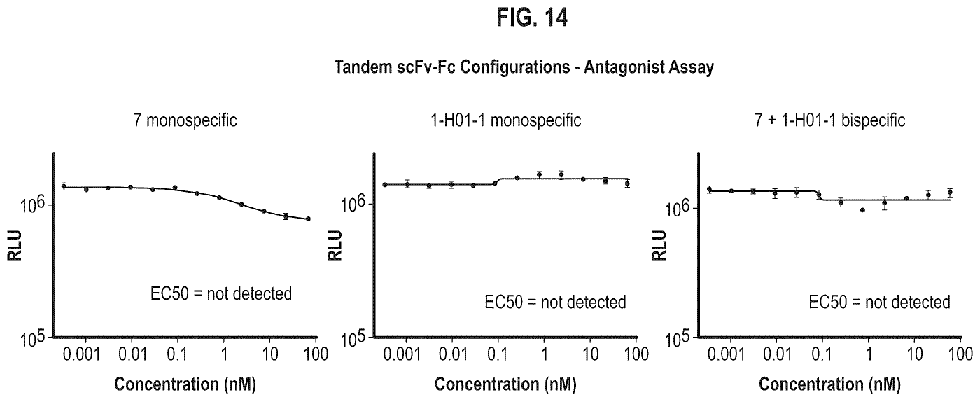

Three tandem scFv-Fc configurations were tested using clones 7 and 1-H01-1: tandem clone 7 monospecific, tandem clone 1-H01-1 monospecific, and tandem clones 7+1-H01-1 bispecific. Differential scanning fluorimetry was used to test the tandem scFv-Fc antibody thermal stability Tm (° C.). FIG. 12 shows the thermal stability for tandem scFv-Fc clones: 7 monospecific, 1-H01-1 monospecific, 7+1-H01-1 bispecific. The three tandem scFv-Fc antibody configurations were tested for their ability to agonize and antagonize the PD-1 pathway with the same in vitro reporter assays used to identify the non-tandem bivalent 7 and 1-H01-1 clones. FIG. 13 shows the PD-1 agonist reporter assay results, FIG. 14 shows the PD-1 antagonist reporter assay results for the three tandem scFv-Fc antibodies tested. In each case, the tandem scFv-Fc configuration with four scFv binding sites shows 2-fold or more PD-1 agonism EC50 improvement relative to the bivalent non-tandem configuration and 3-fold or more PD-1 agonism EC50 improvement relative to the PD1AB6 benchmark. No PD-1 antagonism is observed with the tandem scFv-Fc antibodies tested.

### Example 12: Human Primary Cell Assays for Tandem scFv-Fc Clones

Three tandem scFv-Fc configurations were tested using clones 7 and 1-H01-1: tandem clone 7 monospecific, tandem clone 1-H01-1 monospecific, and tandem clones 7+1-H01-1 bispecific were selected for human primary CD4 T-cell cytokine release and activation marker assays. Human PBMCs were isolated from 6-donors and activated with 5 ug/mL PHA for 48Hr to upregulate PD-1 expression. CD4 T-cells were then purified from the pre-stimulated PBMCs and plated at a uniform density on 96-well plates that were serially coated with 3 ug/mL OKT3 and a titration series of each test article. Supernatants were collected to measure IL-2 at 24Hr and IFN-gamma at 72Hr. CD4 T-cells were collected at 72Hr and analyzed by flow cytometry for PD-1 and CD69 activation biomarkers.

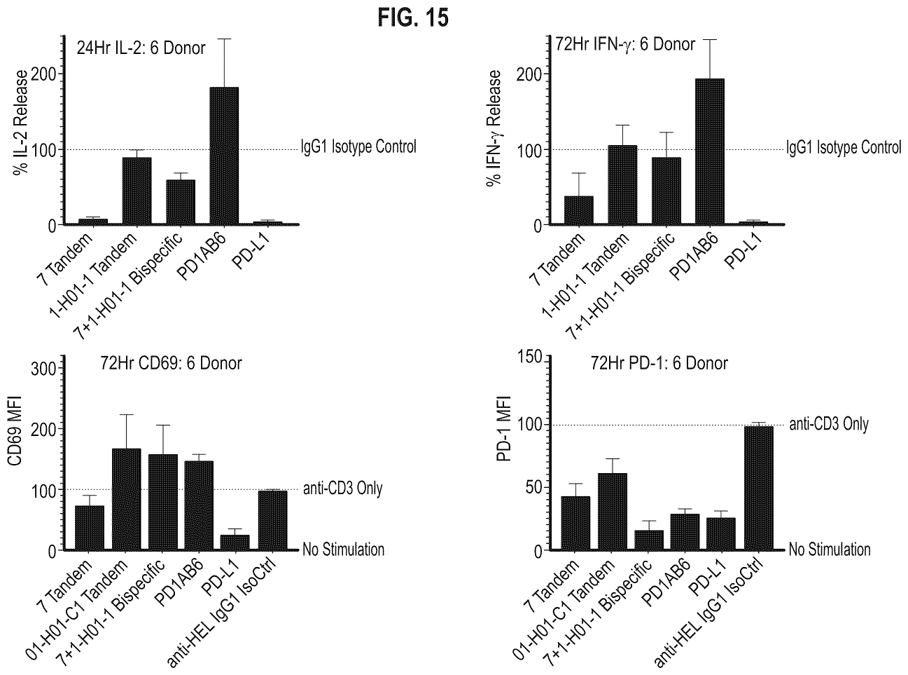

FIG. 15 shows the results of the 6-donor CD4 T-cell cytokine release and activation marker assay. Tandem clone 7 monospecific, tandem clone 1-H01-1 monospecific, and tandem clone 7+1-H01-1 bispecific attenuate IL-2 and IFN-gamma cytokines significantly more than PD1AB6. Tandem clone 7+1-H01-1 bispecific shows significant downregulation of PD-1 expression relative to isotype control and PD1AB6. Tandem clone 7 monospecific shows significant downregulation of CD69 expression relative to isotype control and PD1AB6.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. In embodiments of any of the compositions and methods provided herein, “comprising” may be replaced with “consisting essentially of” or “consisting of”. As used herein, the phrase “consisting essentially of” requires the specified integer(s) or steps as well as those that do not materially affect the character or function of the claimed invention. As used herein, the term “consisting” is used to indicate the presence of the recited integer (e.g., a feature, an element, a characteristic, a property, a method/process step or a limitation) or group of integers (e.g., feature(s), element(s), characteristic(s), propertie(s), method/process steps or limitation(s)) only.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

As used herein, words of approximation such as, without limitation, “about”, “substantial” or “substantially” refers to a condition that when so modified is understood to not necessarily be absolute or perfect but would be considered close enough to those of ordinary skill in the art to warrant designating the condition as being present. The extent to which the description may vary will depend on how great a change can be instituted and still have one of ordinary skilled in the art recognize the modified feature as still having the required characteristics and capabilities of the unmodified feature. In general, but subject to the preceding discussion, a numerical value herein that is modified by a word of approximation such as “about” may vary from the stated value by at least ±1, 2, 3, 4, 5, 6, 7, 10, 12 or 15%.

Additionally, the section headings herein are provided for consistency with the suggestions under 37 CFR 1.77 or otherwise to provide organizational cues. These headings shall not limit or characterize the invention(s) set out in any claims that may issue from this disclosure. Specifically, and by way of example, although the headings refer to a “Field of Invention,” such claims should not be limited by the language under this heading to describe the so-called technical field. Further, a description of technology in the “Background of the Invention” section is not to be construed as an admission that technology is prior art to any invention(s) in this disclosure. Neither is the “Summary” to be considered a characterization of the invention(s) set forth in issued claims. Furthermore, any reference in this disclosure to “invention” in the singular should not be used to argue that there is only a single point of novelty in this disclosure. Multiple inventions may be set forth according to the limitations of the multiple claims issuing from this disclosure, and such claims accordingly define the invention(s), and their equivalents, that are protected thereby. In all instances, the scope of such claims shall be considered on their own merits in light of this disclosure, but should not be constrained by the headings set forth herein.

For each of the claims, each dependent claim can depend both from the independent claim and from each of the prior dependent claims for each and every claim so long as the prior claim provides a proper antecedent basis for a claim term or element.

To aid the Patent Office, and any readers of any patent issued on this application in interpreting the claims appended hereto, applicants wish to note that they do not intend any of the appended claims to invoke paragraph 6 of 35 U.S.C. § 112, U.S.C. § 112 paragraph (f), or equivalent, as it exists on the date of filing hereof unless the words “means for” or “step for” are explicitly used in the particular claim.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A PD-1 agonist antibody or binding fragment thereof, wherein the antibody or binding fragment comprises:
a. a heavy chain variable domain (VH) complementarity determining region (CDR) 1 comprising an amino acid sequence of any one of the following SEQ ID NOs: 10, 16, 22, 29, 32, 36, 37; and
b. a VH CDR2 comprising the amino acid sequence of any one of the following SEQ ID NOs: 11, 17, 23, 30, 33, 35, 38; and
c. a VH CDR3 comprising the amino acid sequence of any one of the following SEQ ID NOs: 12, 18, 24, 25, 34, 39; and
d. a light chain variable domain (VL) CDR1 comprising the amino acid sequence of any one of the following SEQ ID NOs: 13, 19, 26, 40, 42, 46; and
e. a VL CDR2 comprising the amino acid sequence of any one of the following SEQ ID NOs: 14, 20, 27, 31, 43; and
f. a VL CDR3 comprising the amino acid sequence of any one of the following SEQ ID NOs: 15, 21, 28, 41, 44, 45, 47.

2. The antibody or binding fragment of claim 1, wherein the antibody comprises:
a. a VH comprising the amino acid sequence of any one of the following SEQ ID NOs: 1-5, 48-54, and
b. a VL comprising the amino acid sequence of any one of the following SEQ ID NOs: 6-9, 55-61.

3. The antibody or binding fragment of claim 1, wherein the antibody is a monoclonal antibody, a chimeric antibody, or an antibody fragment.

4. The antibody or binding fragment of claim 1, wherein the antibody is fused to an Fc domain of any one of the following: human IgG1, human IgG2, human IgG3, and human IgG4.

5. A method of treating a disease in a subject in need thereof, comprising administering to the subject a therapeutically effective amount of the antibody, wherein the antibody or binding fragment comprises:
a. a heavy chain variable domain (VH) complementarity determining region (CDR) 1 comprising an amino acid sequence of any one of the following SEQ ID NOs: 10, 16, 22, 29, 32, 36, 37; and
b. a VH CDR2 comprising the amino acid sequence of any one of the following SEQ ID NOs: 11, 17, 23, 30, 33, 35, 38; and
c. a VH CDR3 comprising the amino acid sequence of any one of the following SEQ ID NOs: 12, 18, 24, 25, 34, 39; and
d. a light chain variable domain (VL) CDR1 comprising the amino acid sequence of any one of the following SEQ ID NOs: 13, 19, 26, 40, 42, 46; and
e. a VL CDR2 comprising the amino acid sequence of any one of the following SEQ ID NOs: 14, 20, 27, 31, 43; and
f. a VL CDR3 comprising the amino acid sequence of any one of the following SEQ ID NOs: 15, 21, 28, 41, 44, 45, 47.

6. The method of claim 5, wherein the disease is an autoimmune disease, an inflammatory disease, or both.

7. The method of claim 5, wherein the subject is human.

8. A tandem scFv-Fc PD-1 agonist antibody wherein said antibody comprises an scFv1 and an scFv2 binding site in tandem on each antibody arm and wherein said scFv1 and scFv2 are linked by a linker, optionally a flexible linker.

9. The antibody of claim 8, wherein said antibody has a total of four scFv binding sites in a single scFv-Fc formatted antibody.

10. The antibody of claim 9, wherein the scFv1 of each antibody arm comprises a first heavy chain variable domain (VH1) and a first light chain variable domain (VL1); and wherein the scFv2 of each antibody arm comprises a first heavy chain variable domain (VH2) and a first light chain variable domain (VL2).

11. The antibody of claim 10, wherein the VH1 region and the VH2 region each comprises the amino acid sequence of any one of SEQ ID NOS: 1-5 and 48-54, SEQ ID NO: 1 or 53; and wherein the VL1 region and the VL2 region each comprises the amino acid sequence of any one of SEQ ID NOS: 6-9 and 55-61, or preferably SEQ ID NO: 6 or 60.

12. The antibody of claim 11, wherein the linker comprises the following amino acid sequence: GGGGSGGGGSGGGGS (SEQ ID NO: 64).

13. A method of treating a disease in a subject in need thereof, comprising administering to the subject a therapeutically effective amount of an antibody or binding fragment that comprises:
a. a heavy chain variable domain (VH) complementarity determining region (CDR) 1 comprising the amino acid sequence of any one of the following SEQ ID NOs: 10, 16, 22, 29, 32, 36, 37; and
b. a VH CDR2 comprising the amino acid sequence of any one of the following SEQ ID NOs: 11, 17, 23, 30, 33, 35, 38; and
c. a VH CDR3 comprising the amino acid sequence of any one of the following SEQ ID NOs: 12, 18, 24, 25, 34, 39; and
d. a light chain variable domain (VL) CDR1 comprising the amino acid sequence of any one of the following SEQ ID NOs: 13, 19, 26, 40, 42, 46; and
e. a VL CDR2 comprising the amino acid sequence of any one of the following SEQ ID NOs: 14, 20, 27, 31, 43; and
a VL CDR3 comprising the amino acid sequence of any one of the following SEQ ID NOs: 15, 21, 28, 41, 44, 45, 47.

14. The method of claim 13, wherein the disease is an autoimmune disease, an inflammatory disease, or both.

15. The method of claim 13, wherein the subject is human.

16. A nucleic acid sequence encoding the PD-1 agonist antibody or binding fragment that comprises:
a. a heavy chain variable domain (VH) complementarity determining region (CDR) 1 comprising the amino acid sequence of any one of the following SEQ ID NOs: 10, 16, 22, 29, 32, 36, 37; and
b. a VH CDR2 comprising the amino acid sequence of any one of the following SEQ ID NOs: 11, 17, 23, 30, 33, 35, 38; and
c. a VH CDR3 comprising the amino acid sequence of any one of the following SEQ ID NOs: 12, 18, 24, 25, 34, 39; and
d. a light chain variable domain (VL) CDR1 comprising the amino acid sequence of any one of the following SEQ ID NOs: 13, 19, 26, 40, 42, 46; and
e. a VL CDR2 comprising the amino acid sequence of any one of the following SEQ ID NOs: 14, 20, 27, 31, 43; and
f. a VL CDR3 comprising the amino acid sequence of any one of the following SEQ ID NOs: 15, 21, 28, 41, 44, 45, 47.

17. The nucleic acid of claim 16, wherein the nucleic acid sequences are selected from those having at least 95, 96, 97, 98, 99, or 100% sequence identity to SEQ ID NOS: 66 and 67, 68 and 69, 70 and 71, 72, and 73, 74, and 75, 76, and 77, 78 and 79, 80 and 81, 82 and 83, 84 and 85, 86 and 87, 88 and 89, or 90 and 91.

18. The nucleic acid of claim 16, wherein the nucleic acid sequences are selected from those having at least 95, 96, 97, 98, 99, or 100% sequence identity to variable heavy chains selected from 92, 94, 96, 98, 100, 102, or 104; and a light chain selected from SEQ ID NOS:93, 95, 97, 99, 101, 103, 104, or 105.

19. A nucleic acid vector comprising the nucleic acid sequence of claim 16.

20. A host cell comprising the nucleic acid vector of claim 19.
